(19) United States
(12) Patent Application Publication
(10) Pub. No.: US 2006/0240039 A1
(43) Pub. Date: Oct. 26, 2006

(54) Vaccines

(75) Inventors: Adrian Hill, Oxford (GB); Ann C. Moore, Oxford (GB); Claire L. Nicoll, Oxford (GB)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Isis Innovation Limited, Oxford, Oxfordshire (GB)

(21) Appl. No.: 10/560,514

(22) PCT Filed: Jun. 14, 2004
(86) PCT No.: PCT/GB04/02499

§ 371 Date: May 30, 2006

(30) Foreign Application Priority Data
Jun. 13, 2003 (GB) 0313737.9
Jan. 9, 2004 (GB) 0400438.8

(51) Int. Cl. A61K 39/275; A61K 39/285

(52) U.S. Cl. 424/204.1; 424/232.1

(57) ABSTRACT

The present invention relates to a vaccination comprising co-administration of an antigen and viral vector providing immunity at both humoral and antibody levels.

The present invention provides novel methods of vaccination.

Vaccination is a useful tool for combating and preventing disease caused by a number of agents. Such agents may be exogenous, such as viruses, bacteria or parasites. Alternatively, disease-causing agents may be endogenous, such as tumours. Immune responses induced by vaccination may be divided into humoral or antibody responses, and cellular immune responses, such as those mediated by T lymphocytes. A large number of vaccines act by generating protective levels of antibodies.

Antibody-inducing vaccines frequently employ either an entire microorganism, that is often attenuated or heat-killed, or a sub-unit component of the microorganism with an adjuvant. Favoured adjuvants for inducing strong antibody responses include Alum, NP59, CpGs, AS02 and various emulsions.

However, it has recently become clear that cellular immune responses may also be of some use on their own. Various means have been used to induce strong protective cellular immune responses by vaccination.

Known means of generating strong cellular immune responses include DNA vaccination, immunisation with viral vectors or protein particles, the use of recombinant bacteria such as BCG, and heterologous prime-boost immunisation. In fact, heterologous prime-boost immunisation approaches have been found to induce particularly strong effector T cell responses in animals and humans. Such regimes may comprise priming with DNA followed by boosting with recombinant modified virus Ankara (MVA), or priming with recombinant fowl pox and then boosting with recombinant MVA.

In order to combat these more effectively, it is desirable to induce stronger immune responses. However, vaccination methods that generate high level antibody responses differ significantly from those that engender strong cell-mediated or T cell responses. For example, Alum is a useful adjuvant for inducing antibodies but generates weak or negligible CD8⁺ T cell responses. In contrast, heterologous prime-boost immunisation methods have induced strong T cell responses in humans, but only minimal antibody responses. However, immune protection against many diseases can be mediated by either T cells or antibodies at sufficient levels, and optimal protection may be achieved by inducing strong responses of both types. For example, in malaria, it is thought that a pre-erythrocytic vaccine capable of inducing of both high level anti-sporozoite antibodies as well as a strong T cell response against the liver stage parasite would be an ideal method of preventing or treating the disease. However, no vaccination approach currently exists that allows strong responses of each type to be generated. This problem exists not only in malaria, but in a large number of other diseases.

Aventis Pasteur (WO 00/00216) discloses that the use of a Canarypox (ALVAC), in combination with an antigenic peptide, provides an enhanced antibody response to the antigenic peptide. However, there is no indication of any cell-mediated or T cell response in addition to the antibody response.

Accordingly, there is still a need in the art for a method of vaccination that can not only induce a high level antibody response, but also a strong cellular or T cell mediated response. The present invention sets out to overcome this need by providing novel vaccination methods capable of inducing both strong antibody and T cell responses.

Surprisingly, the present inventors have found that co-administration of an antigen with a viral capsid induces both strong antibody and cell-mediated immune responses in a target mammal.

According to one aspect of the present invention, there is provided a vaccine comprising a viral capsid substantially incapable of replication in a patient and at least one antigen for co-administration with said capsid and against which it is desired to obtain an immune response, the vaccine formulated to induce, in a patient to whom the vaccine is administered, both:
an antibody response against the co-administered antigen, and
a T-cell response,
   the at least one antigen and the viral capsid each being formulated separately or together and, when formulated separately, the vaccine being adapted to be administered as either separate formulations or as a mixture thereof

Preferably, the capsid is not ALVAC. Preferably the capsid is an orthopoxvirus such as MVA that have been found to induce stronger T cell responses than avipoxviruses.

Preferably, the viral capsid is a viral vector. Preferably, the viral capsid comprises the simply viral coat with little or no viral polynucleic material. However, it is also preferred that the capsid dose comprise polynucleotides, such as DNA or RNA, encoding viral proteins and even recombinant proteins, for instance markers or antigens as discussed below, provided that the capsid is not capable of replicating. Therefore, the capsid preferably comprises insufficient polynucleotides to replicate and is, therefore, attenuated and/or replication-impaired. Alternatively, the capsid according to the p[resent invention may be viewed as being a capsid comprising any amount of viral or heterologous polynucleotides, provided that the capsid is avirulent.

It is also preferred that the T-cell response is protective.

Preferably, the viral capsid induces a weak or negligible antibody response in the absence of the co-administered antigen.

Preferably, the capsid and the co-administered antigen are formulated for co-administration. The co-administered antigen and the viral capsid may be administered together or separately, and may be administered at the same time or over a period of time, preferably within several days of each other, more preferably on the same day, preferably within 3 hours, particularly within 2 to 3 hours, more preferably within one hour of each other and more preferably substantially together.

Preferably, the co-administered antigen and the viral capsid are admixed and administered as a mixture.

Administration of the co-administered antigen and the viral capsid, whether independently or as a mixture, may be orally or transdermally, but most preferably parenterally. The site of parenteral administration may be intravenously, intramuscularly, subcutaneously, or intradermally, or by any other means known to the skilled person.

Administration of the co-administered antigen with the viral capsid surprisingly stimulates both a humoral and an antibody response to the antigen, thereby providing an immune response on both levels.

The method of administration is, preferably, intradermally, intravenously, intraperitonealy, intramuscularly, orally, intranasally, by aerosol or subcutaneously.

It is preferred that the vaccine is suitable for administration in a homologous prime boost vaccination regimen or, alternatively, a heterologous prime boost vaccination regimen. Preferably the capsid and the co-administered antigen are administered substantially contemporaneously

The nature of the viral capsid is also discussed below. Whilst it is envisaged that the viral capsid is derived from any virus known in the art that is suitable for use therefor, it is particularly preferred that the viral capsid is selected from the group consisting of poxviruses, adenoviruses, retroviruses, and adeno-associated viruses. More preferably, the viral capsid is derived from the herpes viridae family, preferably the varicella viruses, or from the pox viridae family, preferably MVA or NYVAC, ALVAC or a fowlpox virus. However, in one embodiment of the present invention it is preferred that the capsid is not ALVAC.

The nature of the viral capsid is not critical to the present invention. In general the capsid should be able to stimulate a T cell response. Suitable examples of viruses are provided above. It is preferred that the viral capsid is incapable of causing a serious infection in the patient, and it is generally preferred that the virus is incapacitated, such as by heat treatment or attenuation.

In one embodiment of the present invention, it is preferred that empty capsids may be employed, or so called “non-recombinant” capsids that do not comprise polynucleotides encoding an antigen. In this instance, the present inventors have surprisingly shown that a T-cell response to the co-administered antigen can be induced by the presence of the vector. Without being bound by theory, it is thought that the inflammatory response generated by the viral capsid helps to boost the antibody response to the co-administered antigen, by what may be viewed as a “bystander effect”. Thus, the antibody response to the co-administered antigen is aided or adjuvanted by the T-cell response induced by the presence of the vector.

However, it is also preferred that the capsid does encode an antigen, preferably an antigen that is homologous to or from the same infectious pathogen targeted by the vaccine as the co-administered antigen, as discussed below. Therefore, it is preferred that the co-administered and encoded antigens are derived from the same pathogen, i.e. that the vaccine induces an immune response against different antigens from the same pathogen. Accordingly, in a further embodiment, the capsid may comprise polynucleotides encoding a further antigen, referred to herein as the “encoded antigen”. Preferably, this may be homologous to the co-administered antigen. Alternatively, and also preferred, the encoded antigen may be heterologous to the co-administered antigen.

In this instance, the present inventors have surprisingly found that a strong T-cell response, preferably a CD4⁺ or CD8⁺ response, can be induced or generated against the encoded antigen and preserved, despite the fact that an antibody response has been generated. This is surprising as strong antibody responses, generated by many adjuvants and vaccine strategies or regimens, interfere with the generation of strong T-cell responses.

In both the above-mentioned embodiments, it is preferred that the immune response, and in particular the T-cell response, includes a protective or memory aspect, such that the vaccine provides protection against subsequent challenge by the antigen. Preferably, the vaccinee already has specific immunity against the co-administered and/or encoded antigens. In particular, it is preferred that the encoded, when present, antigen comprises at least one CD4⁺ or CD8⁺ epitope suitable for recognition by MHC molecules in the vaccinee.

It is preferred, as mentioned above, that the viral capsid is attenuated, heat-killed or unable to replicate.

It is preferred, as also discussed below, that the antigen (either co-administered or encoded) is derived from M. tuberculosis, Plasmodium sp, influenza virus, HIV, Hepatitis C virus, Cytomegalovirus, Human papilloma virus, bacteria, Plasmodium sp, leishmania parasites or bacteria, preferably Mycobacteria. However, it is also envisaged that the preferred antigen is endogenously derived, for instance a tumour-associated antigen, such that the T-cell and antibody responses induced by the present invention are directed against the tumour.

Preferably, the invention can be applied to carbohydrate and conjugate vaccines. In particular this includes vaccines against H. influenza, pneumococcus and menigococcus. The generation of stronger antibody responses to the carbohydrate component of such vaccines using the current invention is envisaged and it is known in the field that long-lasting T cell memory is required to maintain immunity to these pathogens. Thus the co-administration of either pure polysaccharide vaccines, such as Pneumovax (Merck) or of a conjugate vaccines (e.g. Prevnar) together with a capsid, to generate stronger antibody responses as well as T cell memory, as disclosed in this invention, is envisaged.

Vaccines against Hepatitis B or C are also preferred, in particular, HBsAg.

Preferably, the vaccine induces both an effector T cell response and an antibody response, wherein the effector T cell response is not weaker than that induced by the viral capsid alone, and the levels of antibody induced are not lower than those induced by administration of the antigen alone. In the embodiment where the capsid does not encode a further antigen, the T-cell response is directed towards the co-administered antigen, thereby complementing, and preferably enhancing, the antibody response to said co-administered antigen.

The present invention also provides a method for stimulating both humoral and antibody responses to an antigen, comprising administration of the antigen to a patient in combination with a viral capsid, administration of the capsid and antigen being separately or together.

It is preferred that the antibody response to the co-administered antigen is greater than the antibody response induced by the administration of a vaccine comprising said co-administered antigen and alum, but without the capsid.

It is also preferred that the T cell response induced by the vaccine (to the co-administered or encoded antigen as applicable) is epitope-specific. The T-cell response is preferably a CD4⁺ or preferably a CD8⁺ response, although it is most preferably a combination of the two.

Also provided is a vaccine for inducing an immune response to a co-administered antigen, the vaccine comprising the antigen and a capsid, the capsid in the absence of the antigen inducing a weak or negligible antibody response and in the presence of the antigen inducing a T cell response complementing the antibody response against said antigen, the capsid and the antigen being formulated separately or together.

In one embodiment of the present invention, there is provided a vaccine according to the present invention, wherein the poxviral capsid is an orthopox virus, such as MVA or NYVAC, the presence of an orthopox viral capsid inducing substantially equal ratios of Th-1 and Th-2 Helper T cells.

Alternatively, the poxviral capsid is an avipox virus, such as Fowlpox or Canarypox, the presence of an avipox viral capsid inducing a T cell mediated response, wherein the Th-1 Helper T cell response is greater than the Th-2 response.

There is also provided a vaccine, comprising a polynucleotide and a co-administered antigen, the polynucleotide inducing a T cell response and the co-administered antigen inducing an antibody response, both the T cell and antibody responses being directed to said antigen;

wherein administration of the vaccine comprising the polynucleotide but not the co-administered antigen induces a weak or negligible antibody response;

the polynucleotide and the co-administered antigen being formulated separately or together, being further formulated for co-administration,

the Th-2 Helper T cell-mediated response being greater than the Th-1 response.

It can be seen that in this manner, Th-1 or Th-2 biased responses can be achieved for a particular antigen, so as to enable the administrator of the vaccine to, at least partially, select which, or both, of the Th-1 and/or Th-2 responses to enhance for a particular antigen. The skilled person would be able to determine which of these (Th-1 or Th-2 biased, or both) responses would be most appropriate for the antigen vaccination regimen that he is administering.

The present inventors have shown that prime-boost immunisation with DNA and MVA (DaM induces potent T-cell responses but poor levels of antibody against the encoded HBsAg (Hepatitis B surface Antigen). Conversely, repeat immunisation with Engerix-B, which comprises HBsAg, induced a weak T cell response but greatly increased antibodies to HBsAg compared to D/M.

Surprisingly, however, the present inventors have found that concurrent administration of these vaccines induced both a T cell-mediated response and an antibody response to HBsAg. Further improvements of this co-induction of cell-mediated immunity (CMI) and humoral immunity were sought by combining the viral capsids MVA (Modified Vaccinia Ankara), FP (Fowlpox), ALVAC (avipox virus), NYVAC (attenuated vaccinia virus) and ADV (adenovirus) with the main component of the commercially available Engerix-B vaccine, HBsAg.

So called “non-recombinant” capsids were used which did not encode an antigenic peptide, but did encode a non-antigenic detectable marker, for instance the LacZ gene.

It was found that non-recombinant MVA, FP, ALVAC and NYVAC capsids or vectors adjuvanted co-administered protein leading to greatly enhanced levels of T cells as well as antibodies against the protein.

These results are also supported by experiments in a second model. Vaccines strategies for malaria are often initially tested in the murine model, P berghei. Two vaccines were used and each included either of the T cell inducing capsids FP and MVA, both encoding the P. berghei circumsporozoite protein (the capsids being named FP.CSP and MVA.CSP), and an antibody inducing vaccine, Apv, consisting of Hepatitis B core (HBc) protein and further containing two copies of the P. berghei CSP B cell epitopes (DP₄NPN) adsorbed to alum. Prime-boost immunization with FP.CSP and MVA.CSP induced potent CD8⁺ T cells and no detectable antibodies to the B cell epitope DP₄NPN. Conversely, repeat immunization with Apv induced strong levels of antibodies to DP₄NPN.

Surprisingly, co-administration of FP.CSP, MVA.CSP and Apv lead to specific induction of potent antibodies to both DP₄NPN, and high T cell responses to a CD8⁺ epitope in CSP and HBc. Thus, the present inventors show in two different systems that viral capsids, for instance poxviruses, can enhance both T cell responses and antibody responses to protein thereby demonstrating a method of strongly inducing both cellular and humoral types of immunity against a target antigen.

The dose of poxvirus to be used according to the present invention will be typically in the range of 10⁵ to 10⁷ pfu in small animals such as mice and in the range of 10⁷ to 10¹⁰ pfu in large animals, such as primates and ruminants, including humans. As individual antigens may vary in immunogenicity it may be necessary to undertake dose escalation studies with increasing doses of poxvirus to find the optimal dose. Such dose finding studies are familiar to those skilled in the art.

The present invention further provides vaccines comprising both the co-administered antigen and viral capsid of the invention, as well as a method of inducing both humoral and cell-mediated immune responses in a patient, preferably a human, the method comprising administering a pharmaceutically effective amount of the co-administered antigen and the capsid to the patient

Furthermore the present invention also provides kits comprising preparations the antigen and the capsid, preferably formulated for administration together or separately, preferably as a mixture, as discussed above.

Preferably, the antibody levels induced in the target mammal are greater in the vaccination method according to the present invention than those seen on administration of the co-administered antigen alone. Furthermore, it is also preferred that the T cell response induced as a result of the vaccination method according to the present invention is not less than an order of magnitude less than that induced by administration of the viral capsid alone.

Preferably, the T cell response induced by the present invention is not decreased, and more preferably is increased, compared to the T cell response elicited by the viral capsid alone. Most preferably, both the levels of antibody and the T cell response induced by the present invention are greater than that achieved by administration of the antigen or the viral capsid alone.

Preferably the antigen is proteinaceous. Alternatively, it is also preferred that the antigen is a peptide.

Preferably, the capsid comprises no antigen-encoding nucleic acid. Accordingly, it is preferred that the capsid comprises a nucleic acid encoding a marker such as Lac Z, but no antigenic protein or peptide. Preferably, the capsid is an empty capsid, such as an empty capsid, as discussed above.

The capsid may, optionally, comprise a nucleic acid encoding a protein or peptide. The expressed product of this nucleic acid is preferably an antigen. This expressed antigen may be homologous to the antigen administered with the viral capsid, but may also be heterologous. It is, however, preferred that the antigens are homologous. By this it is meant that they are the same protein or peptide, for instance, or present at least one common epitope. Preferably, the encoded or expressed protein or peptide is recombinant.

It is also preferred that the heterologous antigens are different proteins or peptides, for instance, derived from the same pathogen. For example, the co-administered antigen may be derived from the coat of a new or predicted strain of the influenza virus, whilst the encoded antigen may be derived from a nuclear protein or peptide which is common to all, or a large number of, older strains, the vaccine thus providing immunity against old and new challenges.

Preferably, the vaccine is a combination vaccine for inducing immune responses against heterologous pathogens. For instance, the encoded antigen may be derived from HIV, whilst the co-administered antigen may be derived from Hepatitis B virus.

The subject or patient to which the vaccine is administered is preferably an animal, more preferably a mammal, even more preferably a primate and it is particularly preferred that the patient is a human.

The antigen may be derived from a virus, such as HIV or the Hepatitis B virus, preferably the epitope is derived from a coat or backbone protein. In a preferred embodiment, the antigen comprises the Hepatitis B surface Antigen (HbsAg). However, it is also preferred that the antigen is derived from a parasite, such as a malarial parasite from the Plasmodium family, a bacteria, such as M. tuberculosis, or may even be endogenously derived, for instance a tumour antigen.

In combating HIV, neutralization or opsonisation of HIV-1, for instance, by antibodies combined with the action of CTL suppression of viral replication and induction of cell death of virally infected cells, may have a multiplicative effect on Rₒ, the mean number of cells that are infected by a single infected cell. By reducing Rₒ, the spread of virus and, therefore, viral load, may be limited. This may allow the immune system to focus on rapidly controlling initial infection. Limiting the amount of viral replication may also reduce the virus' opportunity to mutate following immune pressure of specific epitopes which may, therefore, limit viral escape.

The method of vaccination according to the present invention may simply comprise administration of the antigen and the viral capsid. Alternatively, the method may comprise administration of other elements such as adjuvants, such as alum, or other vaccines. The method may also be part of a more complex vaccination regimen, for instance that comprising a homologous, but preferably a heterologous, prime-boost vaccination regimen.

The T cell response induced is preferably a T cell response. Preferably, the response comprises a CD4⁺ T Helper cell response, or a CD8⁺ Cytotoxic T Lymphocytes (CTL) response, and most preferably both, such as a CTL response mediated by T Helper cells, for instance.

WO 00/00216 (Aventis Pasteur, formerly Pasteur Meriux Serums et Vaccins) discloses that the use of a poxvirus, such as Canarypox (ALVAC), in combination with an antigenic peptide, for example an HIV-derived peptide, provides an enhanced antibody response to the antigenic peptide. However, all of the experiments therein relate solely to measuring antibody responses and there is no demonstration of an increased cell-mediated or T cell response.

Clearly, an increased antibody response does not occur spontaneously and in vacuo, but rather is regulated to some extent, depending on the type of antibodies raised, by certain T cell types. However, as is shown in the examples, it is not sufficient to simply measure antibody isotypes to demonstrate that cellular immunity has been induced. Although there is a correlation between Ab and T cell responses, it is weak and inconsistent, such that in many immune responses, one simply cannot be predicted from the other.

However, the Aventis publication does not even consider the prospect that T-cell responses can be raised against the co-administered or encoded antigens and that the antibody response can be complemented in this way. This is shown by the fact that there is no mention of, and in particular no experiment directed towards measuring of, a T-cell response.

Although the present invention may be used in isolation, it may also be combined with other vaccination regimens for combating or preventing the same disease. For example, the encoded and the co-administered antigens may often be different components of the same pathogen, for example the haemaglutinin antigen of influenza A as the antibody target and the nucleoprotein and matrix antigens of the influenza virus as the encoded antigen(s) and, thus, the target of protective T cells. Alternatively, it may be part of a regimen for combating or preventing more than one disease. Accordingly, the present invention may be used in a regimen that elicits an immune response to more than one antigen. For instance, the viral capsid may comprise a nucleic acid that encodes a protein or peptide derived from HIV, whilst the antigen administered according to the present invention is derived from M. tuberculosis. Therefore, in another embodiment of the invention, the viral capsid may encode a protein or peptide that is derived from a different disease-causing agent than the antigen contemporaneously administered with the capsid.

The present inventors have also shown that poxvirus can induce T cell responses at the same time as enhancing antibody responses to the co-administered antigen, the levels of T cell induction being surprisingly high. Therefore, not only is a T-cell response induced, but in one embodiment of the present invention, the response is also surprisingly high.

The inventors have also shown that orthopoxviruses, such as MVA and NYVAC, and fowlpox are useful in adjuvanting antibody induction to a co-administered antigen. Preferably, the use of the capsid, together with the antigen, provides enhanced or greater, and preferably longer lasting, protection than the use of the antigen alone, i.e. in the absence of the capsid.

Accordingly, there is provided a vaccine that induces both an antigen-specific effector T cell response to a virally-encoded antigen and also induces an antibody response to a co-administered antigen.

In the case that the capsid does comprise a polynucleotide encoding an antigen, it is preferred that the T cell response is to the capsid-encoded antigen, rather than the co-administered antigen.

It is also preferred that the capsid, regardless of whether the capsid comprises a polynucleotide encoding an antigen or does not comprise such a polynucleotide, can be used as an adjuvant to enhance the levels of antibodies induced to the co-administered antigen.

Preferably, the virally-encoded antigen is heterologous to the virus and may, as discussed above, originate from a disease-causing agent, such as HIV, a pathogen or a tumour.

It is also preferred that the virally-encoded antigen is a polypeptide, and it is further preferred that said antigen comprises a source of CD4⁺ and CD8⁺ epitopes.

Preferably, the co-administered antigen is not a nucleic acid and may, therefore, preferably also be a polypeptide.

Furthermore, it is preferred that the capsid-encoded antigen and the co-administered antigen are homologous, thereby providing a strong immune response on both cellular and humoral levels.

However, it is also envisaged that that they may be heterologous, such that they may be derived from different disease causing agents, for instance, different pathogens, or one from a pathogen and one from a tumour. The advantage of this approach is that the immune response will be to more than one antigen, thereby allowing for the present invention to be used in so called “combination vaccines”.

The viral capsid used may be adenovirus. However, it is preferred that it is a poxviral capsid. Preferably, the poxvirus is an orthopox virus for instance such MVA or NYVAC, or an avipox, virus for instance fowlpox, ALVAC, the FP9 avipox virus, or derivatives thereof. It is also preferred that that capsid is replication-impaired.

The capsid and the antigen are preferably formulated for co-administration, regardless of whether they have been formulated separately or together, as discussed above. Preferably they are co-administered as a mixture.

The method of administration is, preferably, intradermally, intraepidermally, intravenously, intraperitonealy, intramuscularly, orally, intranasally, by aerosol or subcutaneously.

It is preferred that the vaccine is used to amplify a pre-existing antibody or T cell response, preferably generated by a means other than a prior priming immunization with the same vaccine or vaccines.

Pathogens that may provide suitable antigens are: Hepatitis B virus, influenza virus, HIV, Hepatitis C virus, Cytomegalovirus, Human papilloma virus, Mycobacteria, other bacteria, Plasmodium sp, leishmania parasites, encapsulated bacteria such as pneumococcus and H. influenza. Preferably, the co-administered antigen is particulate.

It is preferred that the induced T cell response is a CD8⁺ T cell response. However, it is also preferred that the T cell response is a CD4⁺ T cell response.

Preferably, the immune responses induced are protective and may be, preferably short-term, but most preferably, long-term.

Preferably, the induced antibody responses to the co-administered antigen are of a greater magnitude than those induced by the same antigen adjuvanted by alum. Furthermore, it is also preferred that the T cell response induced to the co-administered antigen is of greater magnitude than in the absence of the viral capsid.

It is envisaged that the antigen and/or capsid may be mixed or co-formulated with alum or other known adjuvants.

As discussed above, the Aventis publication does not even consider the possibility that T-cell response may be generated. However, we have also shown that cellular responses can be generated to an antigen encoded by a recombinant viral capsid encoding the antigen, and that it is this encoded antigen that is generating the CD4 and CD8 T cell responses. However, the putative cellular immunity referred to in WO 00/00216 relates to the co-administered antigen (i.e. the protein given with the poxvirus). However, we have shown that the cellular response to this may be weak or even absent when the poxvirus does not encode the same antigen.

It is also preferred that the virally-encoded antigen comprises a CD4+ and/or CD8+ T cell epitope against which the vaccinee has a pre-existing specific cellular immune response. Preferably, the immune response was generated by a means other than by immunization with the said recombinant virus, i.e. in a heterologous prime boost regimen.

Also provided is a vaccination method comprising co-administering an antigen together with a vector,

the method inducing both

an antigen-specific T cell response to a poxvirus-encoded antigen, the encoded antigen being heterologous to the poxvirus and comprising a source of CD4⁺ and CD8⁺ epitopes; and
antibodies to the co-administered antigen.

Preferably, the co-administered antigen is not a polynucleic acid.

Also provided is a method of inducing antigen-specific T cell responses in a vertebrate to a poxvirus-encoded heterologous polypeptide antigen comprising a source of CD4⁺ and CD8⁺ epitopes for the vaccinee and inducing antibodies to a co-administered (non-encoded) non-nucleic acid antigen by co-administration of the non-encoded antigen mixed with the said poxvirus.

Further provided is a method of generating an antibody response to an antigen in a vertebrate vaccinee by co-administration, as a mixture, the antigen mixed with an orthopox virus.

Preferably, the orthopox virus is replication-impaired. It is also preferred that the orthopox virus is of the modified vaccinia virus Ankara strain or NYVAC strain or a derivative of either. Preferably, the orthopox virus encodes the co-administered antigen or a homologous sequence. Alternatively, the orthopox virus may encode an antigen that is heterologous to the co-administered antigen.

It is preferred that the orthopox virus encodes a heterologous polypeptide antigen encoding a CD4+ and/or CD8+ T cell epitope against which the vaccinee has a pre-existing specific cellular immune response that was generated by a means other than by immunization with the said recombinant orthopox virus.

The present invention also provides for the use of the present vaccine. In particular, there is provided the use of the vaccine as defined in the present application, in the manufacture of a medicament for use in a method of vaccination. Also provided is the use of a capsid and an antigen in a method of inducing an immune response in a patient. The present invention further provides the use of a capsid in a method of inducing an immune response in a patient, preferably wherein the patient has already been administered with the antigen, or visa versa.

The invention is now described by way of illustration only in the following examples in which the antigen is HBsAg. An Elisa assay was used to measure the anti-HBsAg antibody response. IFN-γ Elispot assays were used to measure T cell responses to whole antigen (HBsAg) or peptide (CD8+ epitope IPQSLDSWWTSL). Statistical analysis was provided using the SPSS 11.0 program to perform Mann-Witney U tests (non-parametric test, 2 independent samples). Exact significance [2*1-tailed].

A number of commercially available vaccines are available for Hepatitis B that induce protective levels of antibodies. However, not all patients successfully sero-convert to protective levels (Alper F E et al 1995 exp clin immunogenet 12:171-81). These vaccines induce high levels of anti-HBsAg antibody but a minimal specific effector T-cell response as measured, for example, by the widely used interferon-gamma ELISPOT assay. As discussed above, one of the current challenges facing vaccinology is the identification of means to induce strong humoral and cellular response concurrently. Both responses may be of value in preventing and treating a number of diseases including hepatitis B virus infection. Inducing both responses may provide protection to the percentage of the population who fail to sero-convert to the standard alum-adjuvanted commercially available vaccine (for example, the Engerix B product), and in the treatment of persistent hepatitis B virus infection.

DNA and MVA has been used for a number of years in laboratory animals and in many human clinical trials as a vector for the antigen or antigens of interest (Schneider et al 1999 imm. Rev, Hanke, Vaccine. May 6 2002: 20(15): 199-8, Matteo 1999 J immunol 163:4058) in order to induce strong effector CD4+ and CD8+ T cell responses.

### Experiment 1

Aim:

Induction of a strong humoral response to HBsAg while still maintaining a strong cellular response by combining a vaccine to induce protective antibody levels (Engerix-B contains HBsAg and PreS regions of HBV adsorbed to Alum) with a T-cell inducing regime (DNA prime and MVA boost, both containing HBsAg and PreS regions of HBV).

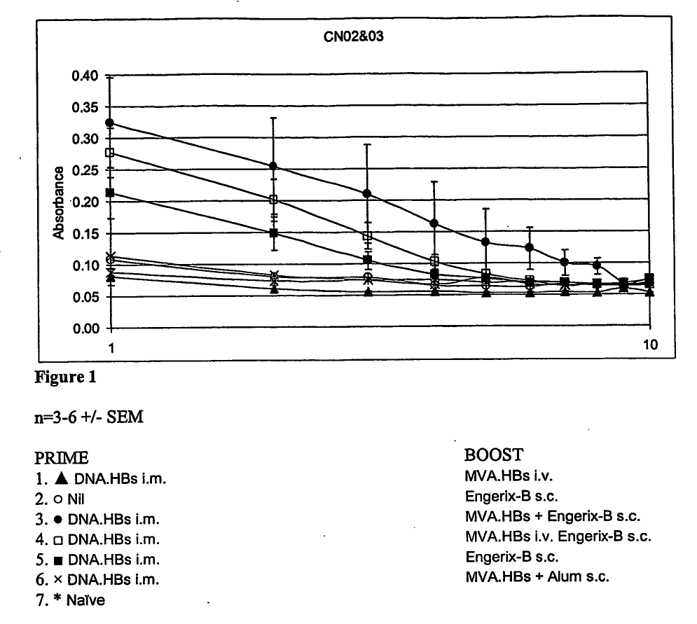

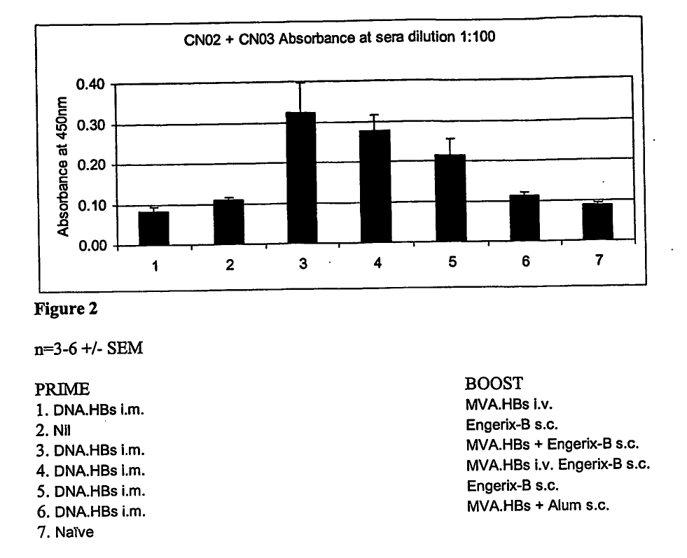

1.1 Antibody Responses (FIGS. 1 and 2)

MVA.HBs Adjuvants Engerix-B

DNA.HBs prime followed by MVA.HBs mixed with Engerix B and administered s.c. produced the strongest antibody response. The next best immunisation regime was DNA.HBs prime followed by MVA.HBs i.v. and Engerix B s.c.

This indicated that, surprisingly, MVA.HBs when mixed with Engerix-B (group 3) was further adjuvanting the HBsAg in the Engerix-B vaccine leading to a stronger antibody response than was observed when the Engerix-B and MVA.HBs were administered separately (group 4). This is further supported by DNA.HBs priming followed by Engerix-B boosting (group 5) which gave lower responses than when MVA.HBs was included in the boost (group 3).

DNA.HBs Priming Increases Antibody Responses to Engerix-B

DNA.HBs however, was in part responsible for the increase in antibody responses. One shot of Engerix B at week 2 (no immunization at prime) gave significantly lower antibody responses than DNA.HBs priming followed by boosting with Engerix-B with or without MVA.HBs (s.c. or i.v) (p=0.026, 0.002, 0.002 respectively).

DNA.HBs priming followed by MVA.HBs boosting gave the lowest antibody response, not significantly different from unimmunised animals (p=0.931).

MVA.HBs boosts antibody response when administered at the same time as Engerix B either at the same or different sites.

DNA.HBs priming increases the antibody response to a single shot of Engerix B given 2 weeks following priming.

1.2 T-Cell Responses

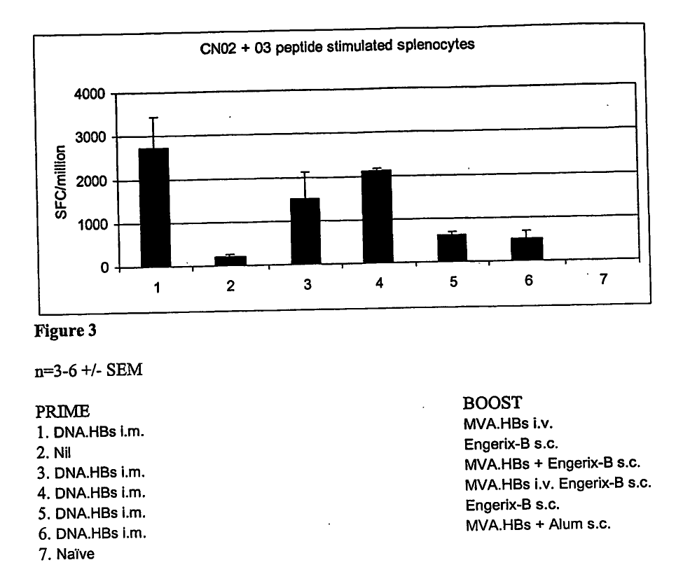

1.2.1 Peptide Stimulated Splenocytes (FIG. 3)

DNA.HBs prime and MVA.HBs boost gave the strongest T cell responses but not significantly different to DNA.HBs priming followed by boosting with Engerix-B and MVA.HBs(s.c. or i.v) (p=0.343 both s.c. and i.v.).

DNA.HBs priming followed by boosting with Engerix-B and MVA.HBs i.v was significantly better than DNA.HBs priming followed by Engerix-B boosting (p=0.029). This suggests that MVA.HBs is responsible for inducing high levels of specific T-cells and that this response is not significantly reduced when combined with Engerix-B.

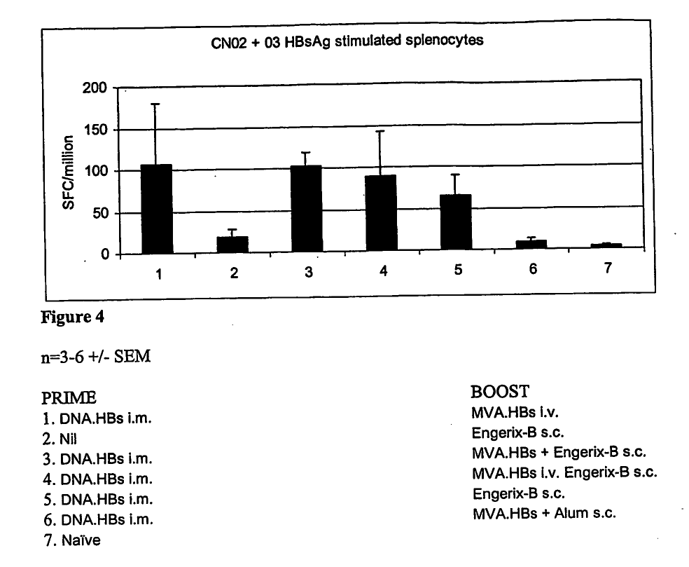

1.2.2 HBsAg Stimulated Splenocytes (FIG. 4)

A similar patter to peptide stimulated splenocytes is seen upon HBsAg stimulation, although much lower overall response levels, probably because processing and presentation of the entire protein is incomplete; no processing is required for peptide presentation.

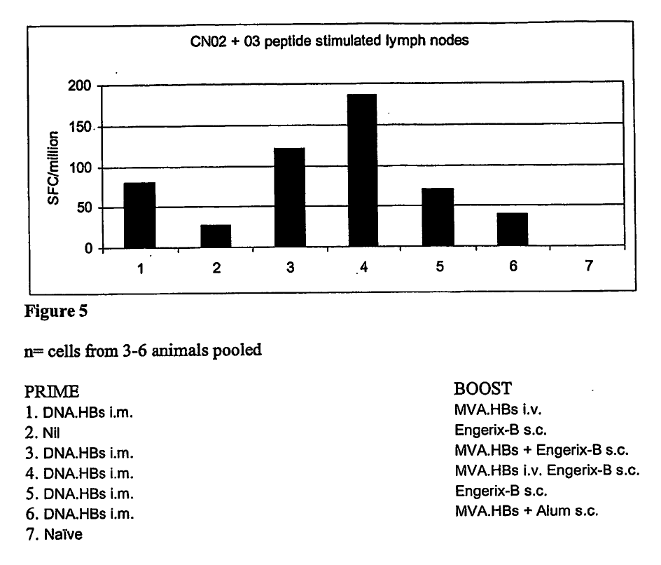

1.2.3 Peptide Stimulated Lymph Nodes (FIG. 5)

Again, a similar pattern to peptide stimulated splenocytes. Cells from cervical and axial lymph nodes for each group were pooled. The strongest response was seen in DNA.HBS primed and MVA.HBs i.v. and Engerix-B boosted animals.

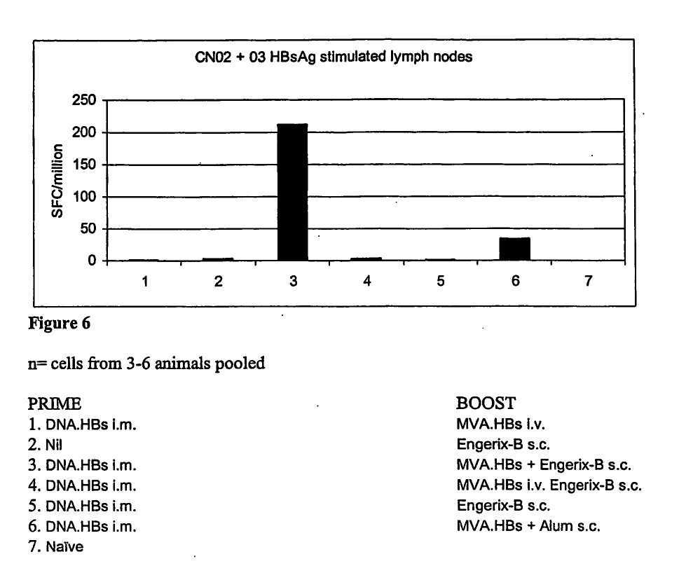

1.2.4 HBsAg Stimulated Lymph Nodes (FIG. 6)

Responses to whole antigen was well below 50 SFC/million except for animals primed with DNA.HBS and boosted s.c. with MVA.HBs and Engerix-B where cell numbers were 212 SFC/million.

### Experiment 2

To further increase Ab levels by including Engerix-B in the prime and still maintain T-cell responses. To establish whether the enhancement still apply when the route of MVA administration is changed to intradermal (id) applies and to bring it closer to the subcutaneous (sc) immunisation site of Engerix-B, so that both immunizations share the same draining lymph node. To establish whether an MVA vector not encoding an antigen (MYA.LacZ) boosts responses to HBsAg

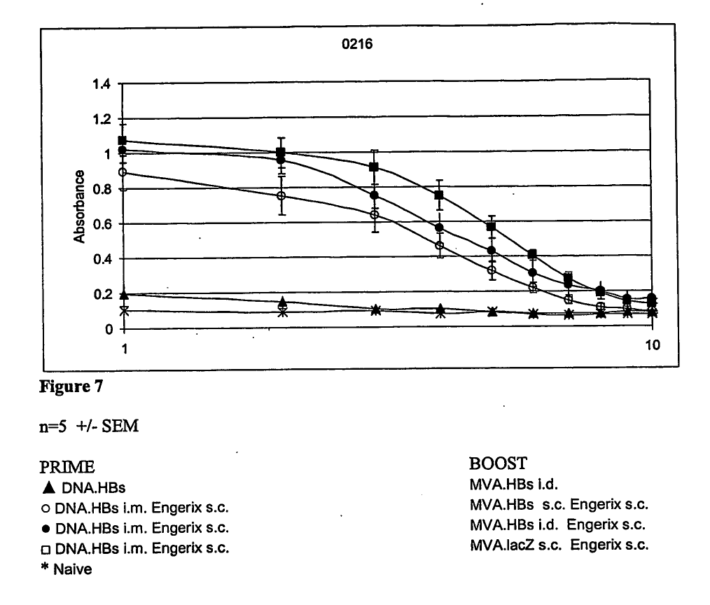

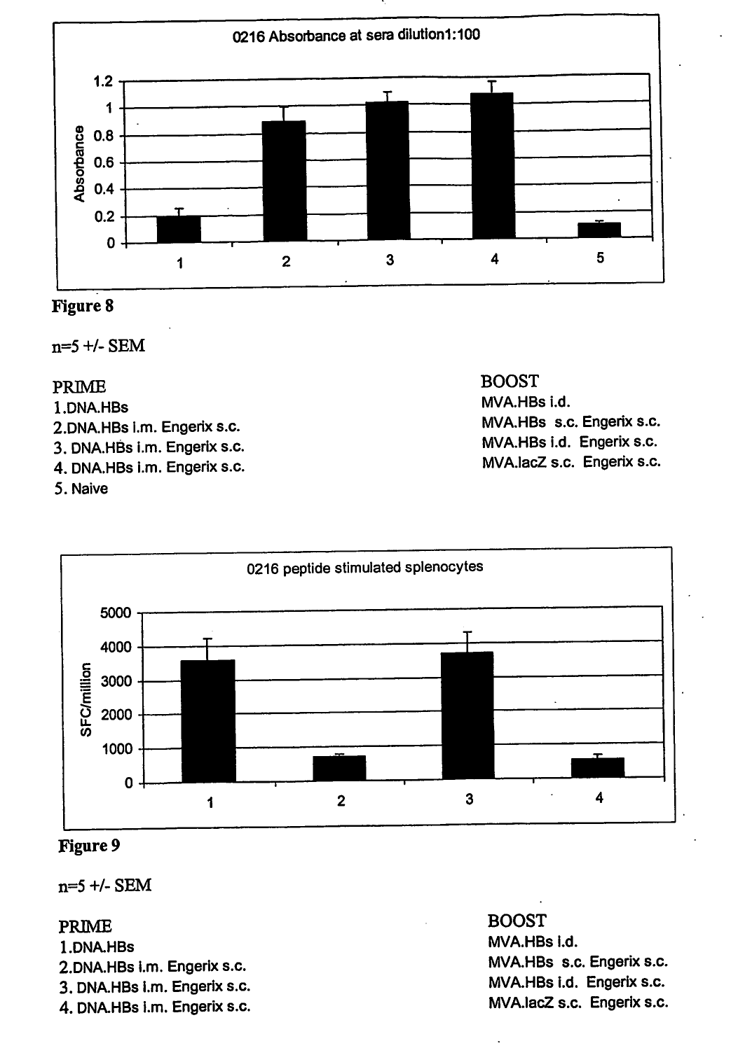

2.1 Antibody Responses (FIGS. 7 and 8)

MVA.LacZ and MVA.HBs boosts antibody responses equally

DNA.HBs and Engerix-B priming combined with a boost of Engerix-B and either MVA.HBs(i.d or s.c) or MVA.LacZ s.c. increased antibody responses 10-fold compared to any immunisation regime in Experiment one.

The antibody responses to these three regimes were not significantly different from each other and it can therefore be concluded that MVA boosts antibody responses against BHsAg by acting as an adjuvant.

2.2 T-cell Responses

2.2.1 Peptide Stimulated Splenocytes (FIG. 9)

DNA.HBs prime followed by MVA.HBs boost along with DNA.HBs and Engerix-B priming combined with a boost of Engerix-B s.c. and MVA.HBs i.d. give equally strong T-cell responses to peptide. However if MVA.HBs or MVA.LacZ is given s.c. with Engerix-B following DNA.HBs and Engerix-B priming this cellular response is abrogated. Therefore in order for strong cellular responses to be maintained the MVA needs to express the antigen present in the protein immunisation, in this case HBsAg. Priming with Engerix-B and DNA.HBs then boosting with MVA.HBs i.d. and Engerix-B s.c. significantly increases T-cell responses to peptide when compared with Exp 1 regime of DNA.HBs priming followed by boosting with MVA.HBs i.v. and Engerix-boost (p=0.004).

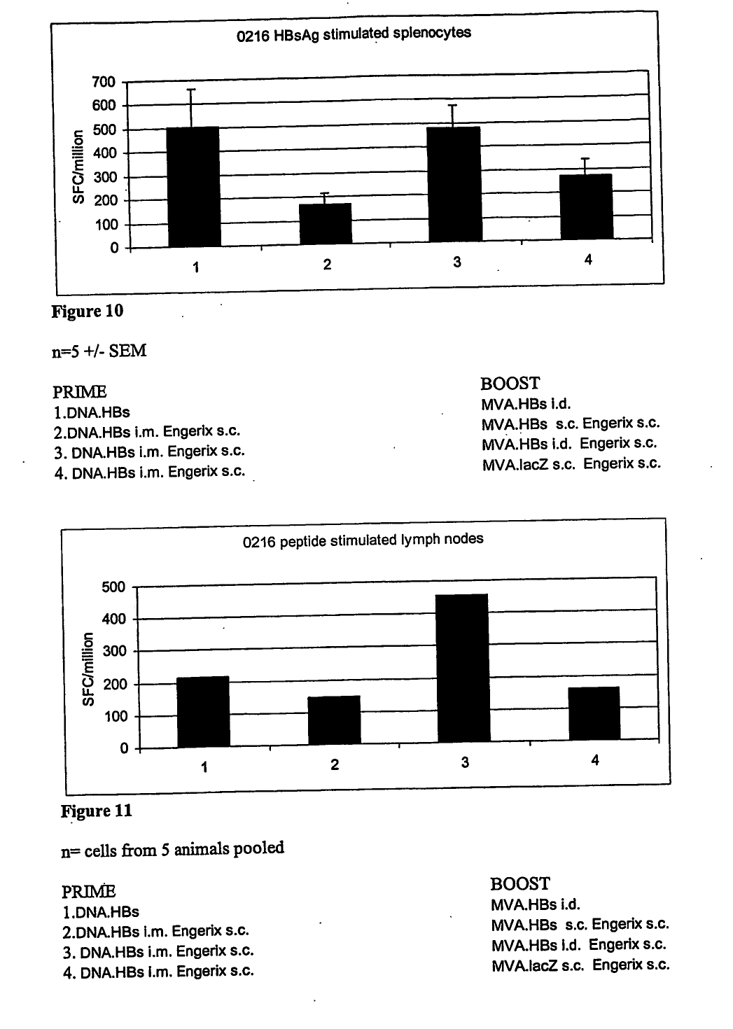

2.2.2 HBsAg Stimulated Splenocytes (FIG. 10)

A similar pattern to that of peptide-stimulated splenocytes is seen upon HBsAg stimulation of splenocytes. The numbers are a lot lower than peptide stimulation. However, they are greater than those seen in experiment one.

2.2.3 Peptide Stimulated Lymph Nodes (FIG. 11)

As with Exp 1 when MVA.HBs is given at an alternate site to the Engerix boost the t-cell response is elevated in comparison to other immunisation regimes.

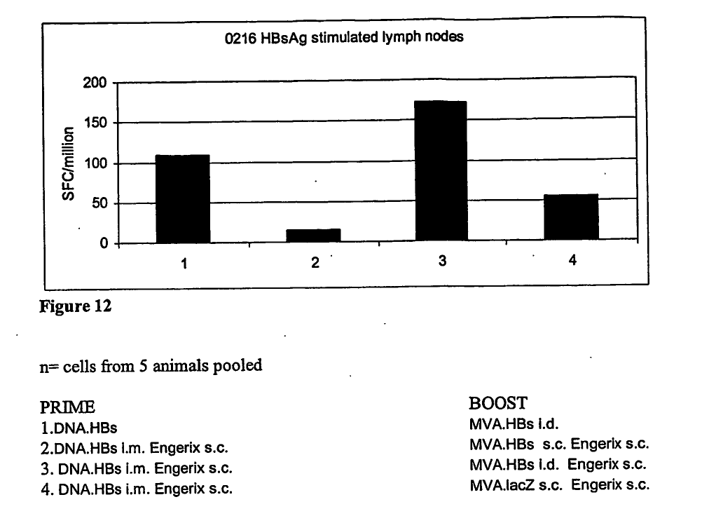

2.2.4 HBsAg Stimulated Lymph Nodes (FIG. 12)

Same pattern as peptide stimulated LN's, but half the numbers. Again a much higher response(11 fold) when MVA.HBs is given i.d. compared to s.c. with Engerix-B. Although, in Exp 1 DNA.HBs prime followed by MVA.HBS and Engerix-B boost s.c. is the only regime that gives good responses to HBsAg stimulated LN's.

### Experiment 3

Aim:

To establish whether a good cellular and Ab responses can be achieved in the absence of Alum using a homologous boost.

The same immunising agent and route was used for prime and boost.

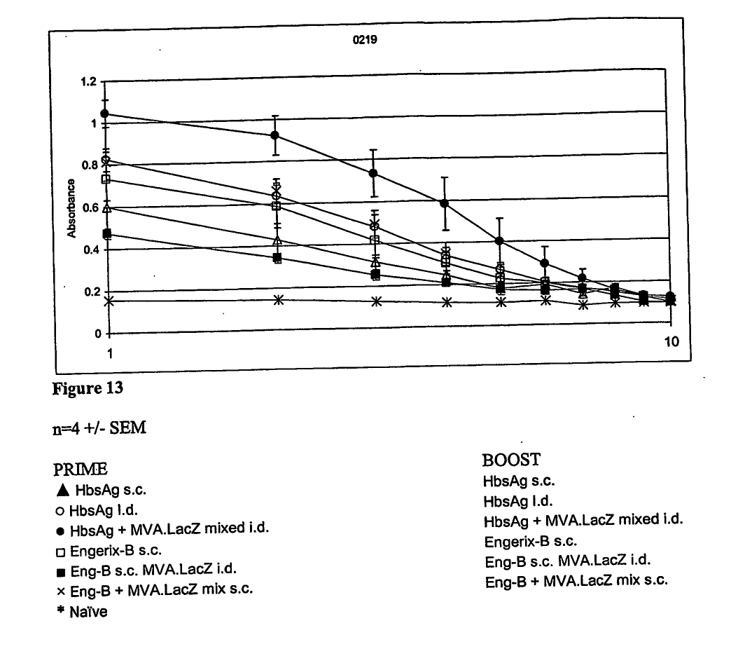

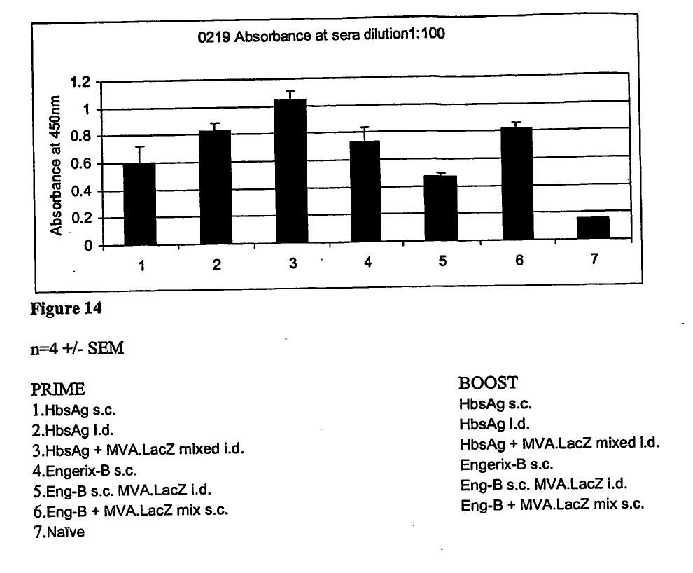

3.1 Antibody Responses (FIGS. 13 and 14)

Highest level of antibodies were achieved with HBsAg mixed with MVA.LacZ and administered i.d. Levels were not significantly different to Exp 2 immunisations where DNA.HBs priming was followed by boosting with Engerix-B and MVA.HBs (i.d. or s.c.) or MVA.LacZ s.c. (p=0.905, 0.19 and 0.905 respectively).

Two immunisations with Engerix-B produced lower antibody responses than HBsAg mixed with MVA.LacZ administered i.d (p=0.057).

Homologous immunisation with HBsAg i.d. alone was not significantly different from Engerix-B alone or HBsAg and MVA.LacZ given i.d., (p=0.686 and 0.114 respectively).

Engerix-B given with MVA.LacZ s.c (prime and boost) was significantly lower in two groups from Exp 1 that received DNA.HBs and Engerix-B priming combined with a boost of Engerix-B and either MVA.HBs i.d. or MVA.LacZ s.c (p=0.032 both). But not if the MVA.HBs was given s.c. (p=0.556)

This indicates that DNA.HBs priming increases the antibody response to HBsAg.

High levels of antibody can be induced by priming with DNA.HBs and Engerix-B then boosting with MVA.LacZ or MVA.HBs and Engerix-B. These levels are comparable to those induced by two homologous immunisations of HBsAg and MVA.LacZ i.d.

3.2 T-Cell Responses

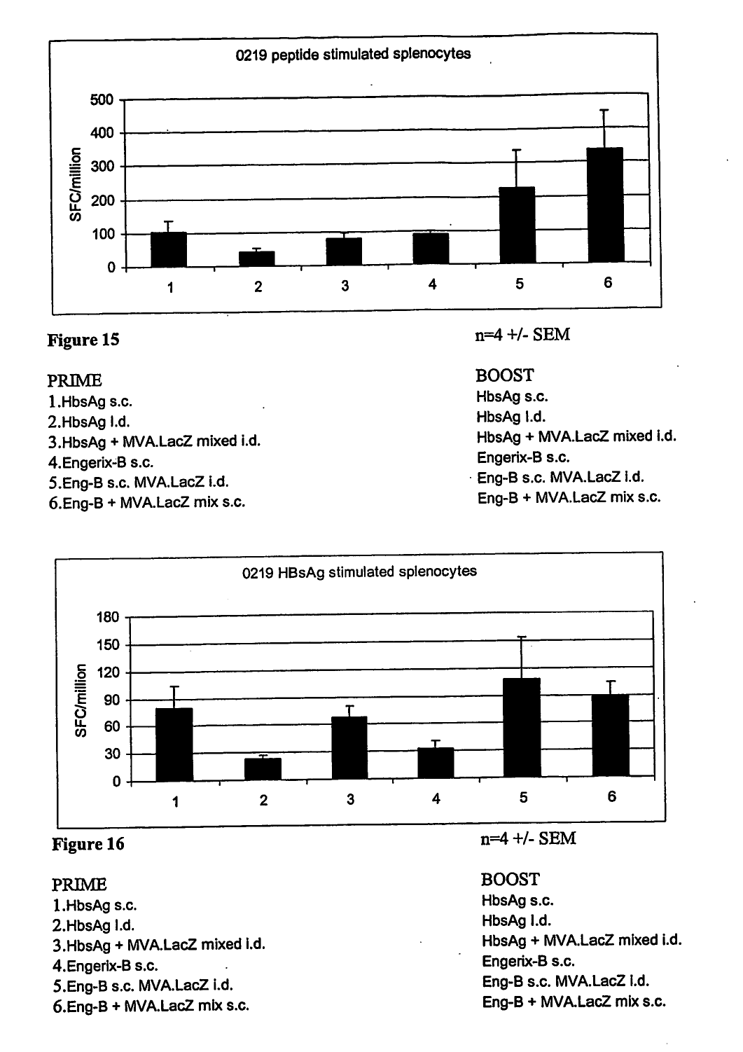

3.2.1 Peptide Stimulated Splenocytes (FIG. 15)

T-cell responses to all vaccine regimes were very low(all well below 500 SFC/million) when compared to the best regimes of other Experiments.

Surprisingly, homologous prime/boost of Engerix-B mixed with MVA.LacZ and administered s.c yielded similar numbers to the Spec2 ??? regime of DNA.HBs priming followed by boosting with Engerix-B and MVA.LacZ s.c. (average of 336+/−113 SEM and 511+/−173 SEM SFC/million respectively). This suggests that DNA.HBs combined with Engerix-B priming is not enough to increase cellular responses to HBsAg when the MVA combined with Engerix-B is non-specific, in this case MVA.LacZ given either s.c or i.d. The MVA administered at the time of boosting needs to contain HBs in order to attain high levels of specific T-cells.

3.2.2 HBsAg Stimulated Splenoctyes (FIG. 16)

All below 150 SFC/million

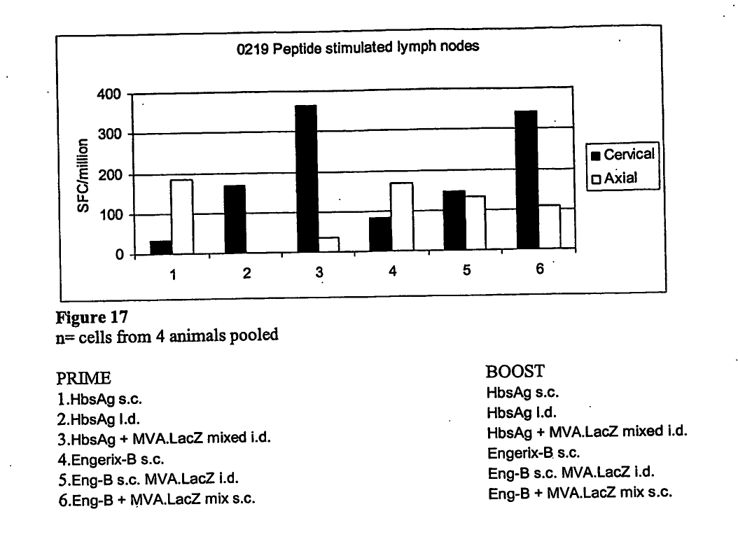

3.2.3 Peptide Stimulated Lymph Nodes (FIG. 17)

Kept Axial and Cervical Lymph nodes separately. s.c. immunisations more likely to predominantly drain to the Axial LN's, i.d immunisations more likely to predominantly drain to cervical L.N's. HBsAg alone s.c gives higher responses in Axial L.N's. When given i.d. Cervical LN's have higher responses, equal to level in Axial LN's following s.c. immunisation.

Favourable regimes were either HBsAg mixed with MVA.LacZ and given i.d. or Engerix-B mixed with LacZ and given s.c.(363 and 343 respectively) in cervical LN's. Engerix-B given s.c. and MVA.LacZ given i.d. produced almost equal numbers of spots in cervical and axial LN's (147 and 132 respectively) and was similar in number to Engerix-B alone(83 cervical and 167 axial). This suggested that MVA.lacZ did not adjuvant Engerix-B in LN's when given i.d. but did in cervical LN's if mixed with Engerix-B and given s.c, therefore scenario the antigen of interest may need to be immunised at the same site as the MVALacZ to be adjuvanted by it.

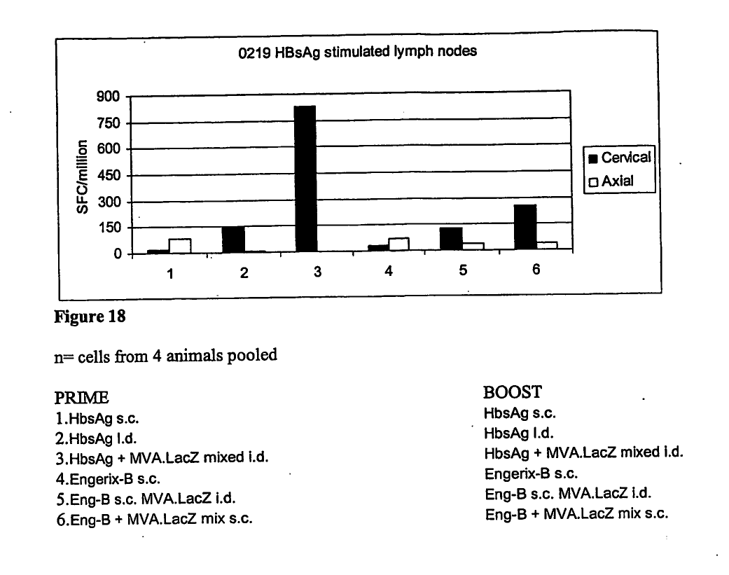

3.2.4 HBsAg Stimulated Lymph Nodes (FIG. 18)

Similar pattern to peptide stimulation of LN's except even larger response in cervical LN's following i.d. immunisation of HBsAg mixed with MVA.LacZ(828 SFC/million).

### Conclusions from Experiments 1-3

DNA priming was shown to be important for inducing cellular and to a lesser extent antibody responses.

Exp 1 showed DNA.HBs priming followed by boosting with Engerix-B and MVA gave good cellular responses but low Ab levels.

Exp 2 showed that by including an Engerix-b prime with DNA then boosting with Engerix-B and MVA gave high T-cell and antibody levels

Exp 3 showed homologous prime/boost with MVA and HBsAg gave strong Ab responses but very low T-cell responses.

Antibody Responses

a) MVA.LacZ and MVA.HBs can adjuvant the protein HBsAg to induce high levels of specific antibody against HBsAg.

b) DNA.HBs priming increases the antibody response to BBsAg when Engerix-B s.c. is concurrently used to prime followed by boosting with Engerix-B and MVA.lacZ s.c. (Experiment 3 group 4 vs Spec 3 group 6)

c) MVA.LacZ adjuvants Engerix-B to produce high antibody levels when both are mixed and immunised s.c. (experiment 3, group 5 vs group 6)

T-Cell Responses

a) DNA.HBs priming is required to induce high levels of specific T-cells. (Exp 3 vs Exp 1 & 2)

b) Following DNA priming highest T-cell responses are achieved by administering MVA.HBs i.v. or i.d, not s.c. (Spec 2)

c) Engerix-B used at prime with DNA HBs increases T-cell responses when boosted with MVA.HBs iv. and Engerix-B s.c. (Spec 2 group 3 vs Spec 1 group 4)

A favourable Strategy for Inducing Antibody and T-cells to D ate:

Prime: DNA.HBs i.m. and Engerix-B s.c.

Boost: MVA.HBs i.d. and Engerix-B s.c.

T-cell responses: Not significantly different (P=0.61) from our ‘gold standard’ T-cell inducing regiment DNA.HBs i.m. prime and MVA.HBs i.v. or i.d. boost.

Antibody responses: Higher antibody levels than the ‘gold standard’ of homologous prime/boost with Engerix-B, of marginal statistical significance (p=0.06)

MVA may direct the immune system towards epitopes not normally targeted in current vaccine regimes thus overcoming the problem of non-responders.

### Experiment 4

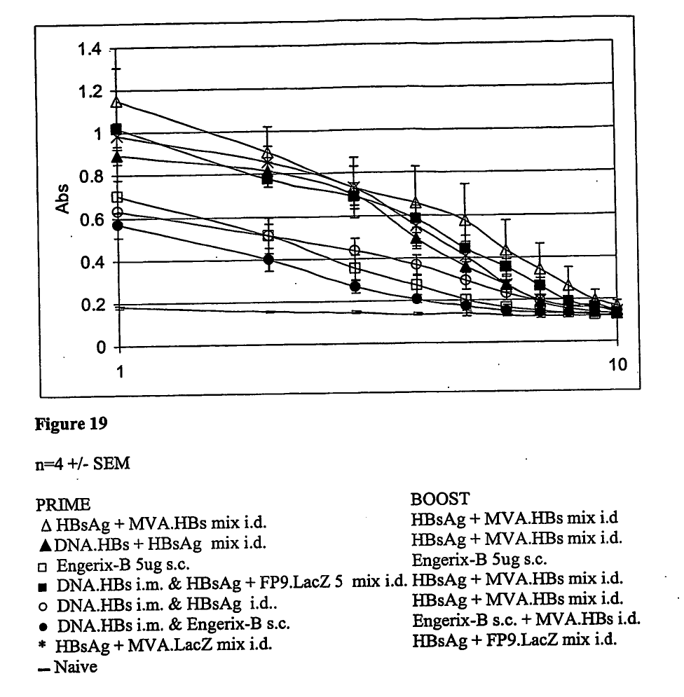

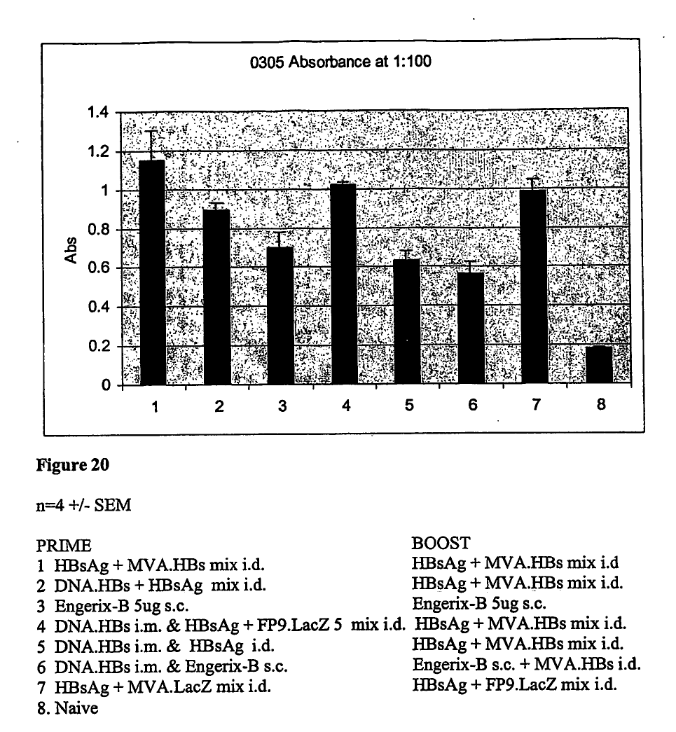

Aim: to retain HBsAg and MVA boost giving a good Ab responses, whilst improving the prime to restore cellular responses. Furthermore, an aim of this experiment was to establish whether the strong cellular response restored if MVA.HBs is used to adjuvant HBsAg at prime and boost, and whether FP.LacZ has an adjuvant activity for antibody induction. 4.1 Antibody Responses (FIGS. 19 and 20)

4.2 T-cell Responses

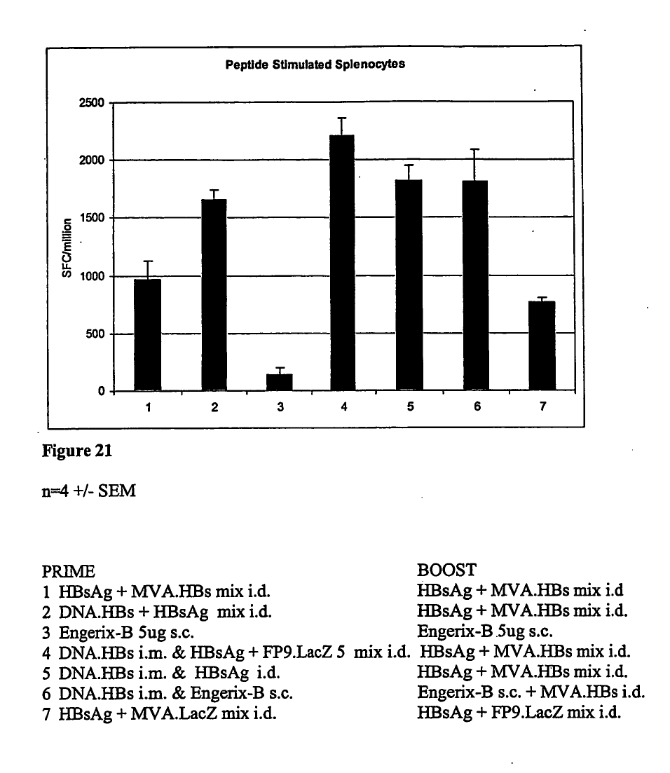

4.2.1 Peptide Stimulated Splenocytes (FIG. 21)

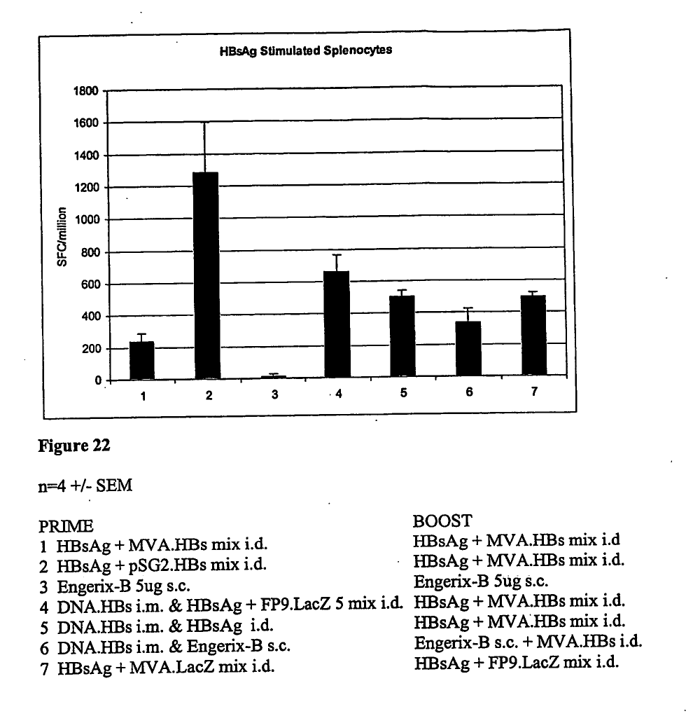

4.2.2 HBsAg stimulated splenocytes (FIG. 22)

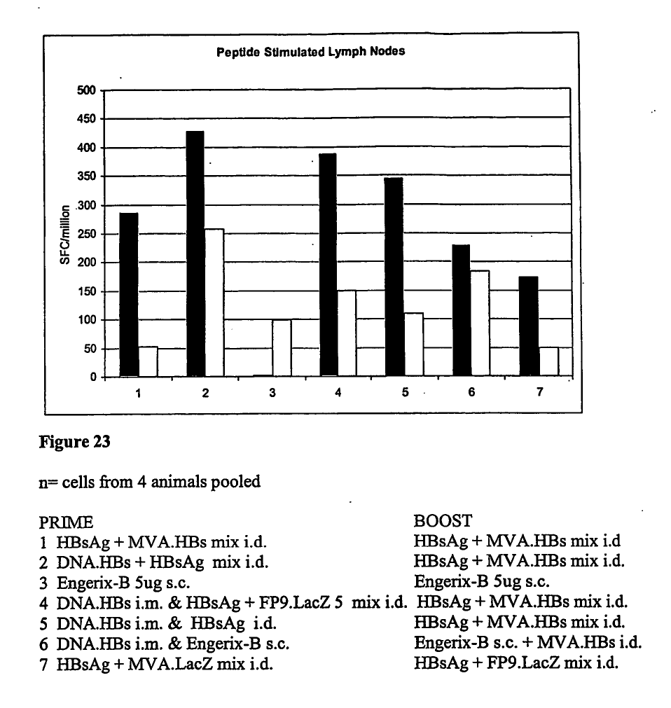

4.2.3 Peptide Stimulated Lymph Nodes (FIG. 23)

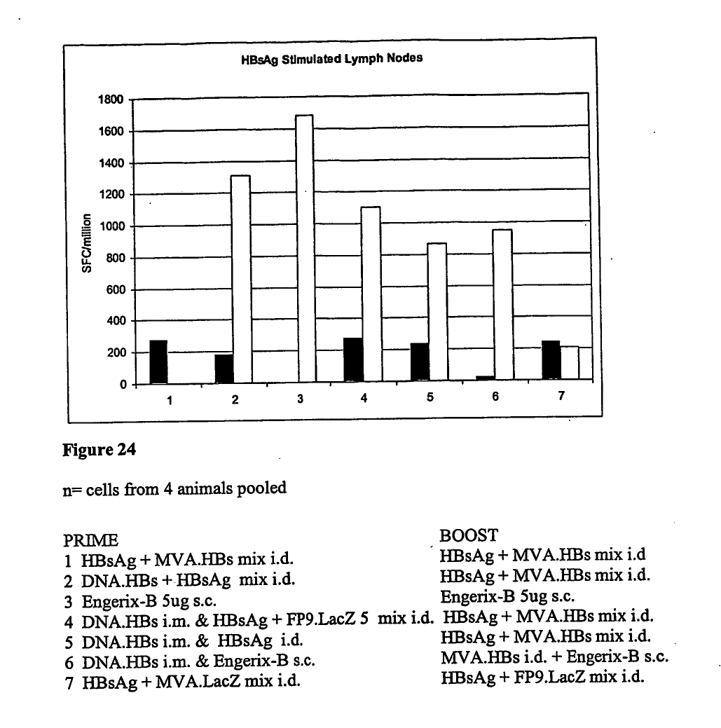

4.2.4 BsAg Stimulated Lymph Nodes (FIG. 24)

### Experiment 4 demonstrates that

1. FP9 has adjuvant activity for antibody induction to the co-administered antigen—compared groups 4 and 5.

2. A heterologous prime-boost regime is required for optimal T cell induction; compare T cell responses in spleen in groups 1 and 2.

### Summary of Results from Experiments 1-4

For ease of reference, the results of experiments 1-4 are compared, using the following key:

| PRIME | BOOST |
|---|---|
| 1. DNA.HBs i.m. | MVA.HBs i.v. |
| 2. Nil | Engerix-B s.c. |
| 3. DNA.HBs i.m. | MVA.HBs + Engerix-B s.c. |
| 4. DNA.HBs i.m. | MVA.HBs i.v. Engerix-B s.c. |
| 5. DNA.HBs i.m. | Engerix-B s.c. |
| 6. DNA.HBs i.m. | MVA.HBs + Alum s.c. |
| 7. DNA.HBs | MVA.HBs i.d. |
| 8. DNA.HBs i.m. Engerix s.c. | MVA.HBs s.c. Engerix s.c. |
| 9. DNA.HBs i.m. Engerix s.c. | MVA.HBs i.d. Engerix s.c. |
| 10. DNA.HBs i.m. Engerix s.c. | MVA.lacZ s.c. Engerix s.c. |
| 11. HbsAg s.c. | HbsAg s.c. |
| 12. HbsAg I.d. | HbsAg I.d. |
| 13. HbsAg + MVA.LacZ mixed i.d. | HbsAg + MVA.LacZ mixed i.d. |
| 14. Engerix-B s.c. | Engerix-B s.c. |
| 15. Eng-B s.c. MVA.LacZ i.d. | Eng-B s.c. MVA.LacZ i.d. |
| 16. Eng-B + MVA.LacZ mix s.c. | Eng-B + MVA.LacZ mix s.c. |
| 17. HBsAg + MVA.HBs mix i.d. | HBsAg + MVA.HBs mix i.d |
| 18. HBsAg + pSG2.HBs mix i.d. | HBsAg + MVA.HBs mix i.d. |
| 19. Engerix-B 5 ug s.c. | Engerix-B 5 ug s.c. |
| 20. DNA.HBs i.m. & HBsAg + | HBsAg + MVA.HBs mix i.d. |
| FP9.LacZ 5mix i.d. | |
| 21. DNA.HBs i.m. & HBsAg i.d. | HBsAg + MVA.HBs mix i.d. |
| 22. DNA.HBs i.m. & Engerix-B s.c. | MVA.HBs i.d. + Engerix-B s.c. |
| 23. HBsAg + MVA.LacZ mix i.d. | HBsAg + FP9.LacZ mix i.d. |

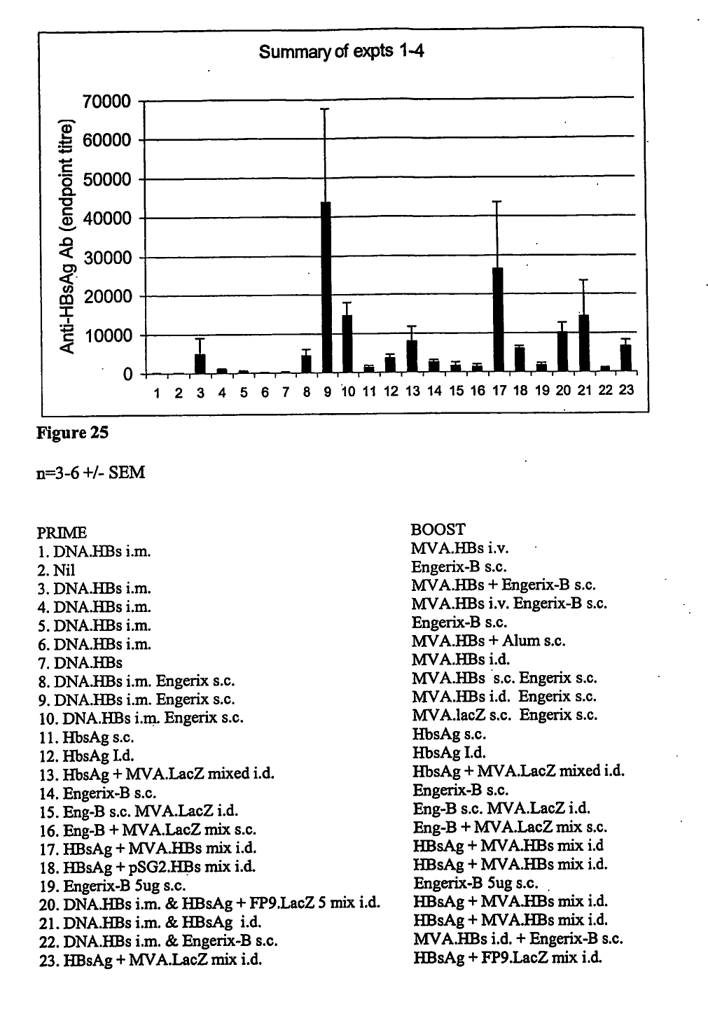

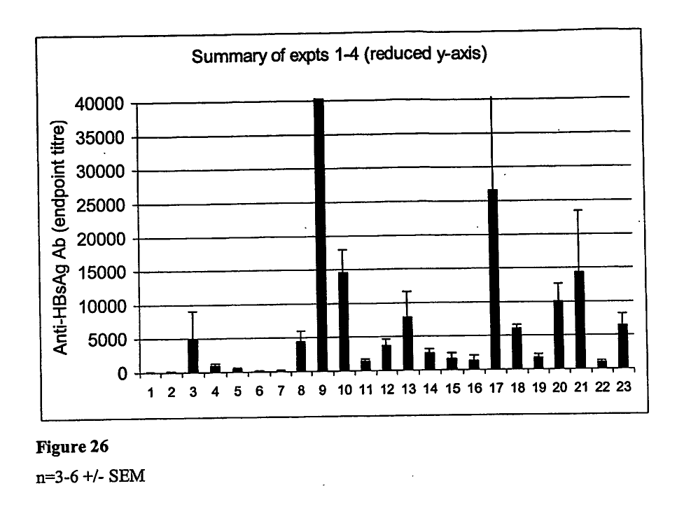

Antibody Responses (FIGS. 25 and 26)

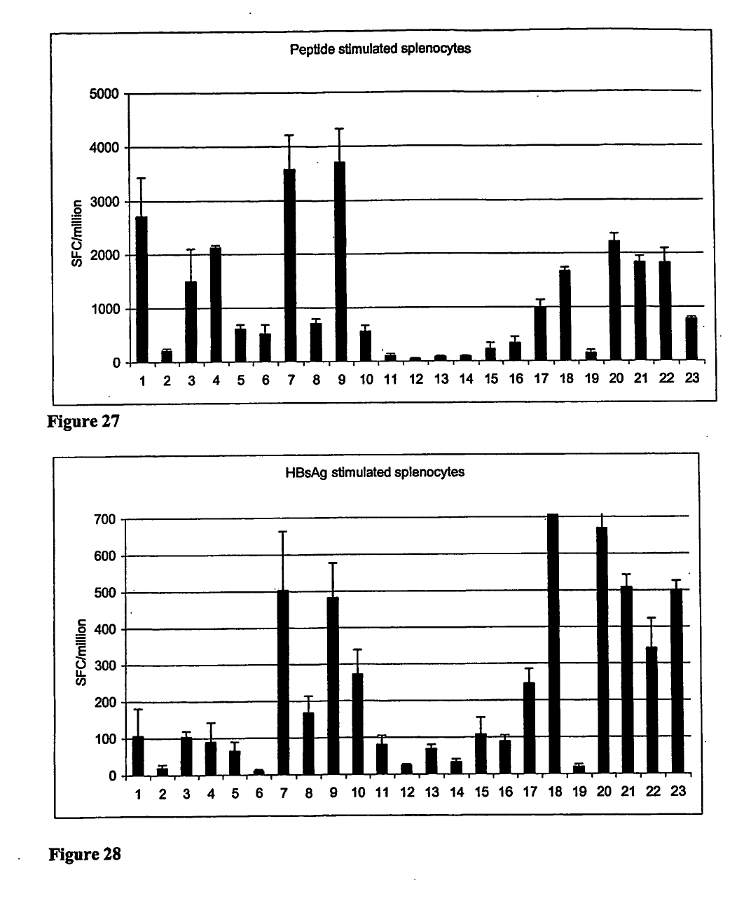

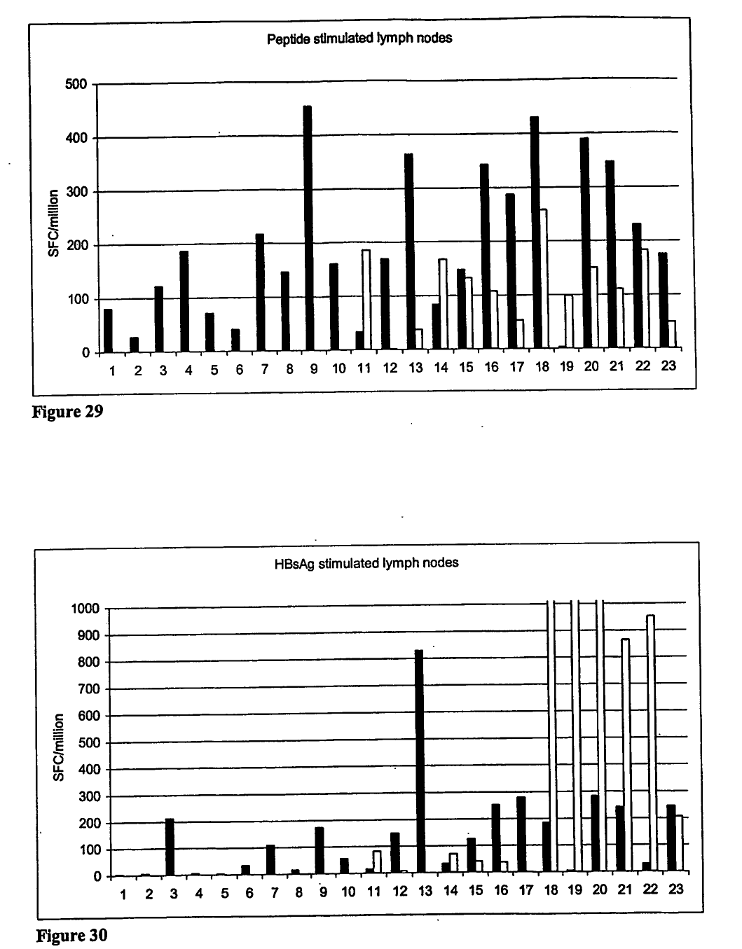

Cellular Responses (FIGS. 27 to 30)

### Experiment 5

Favourable antibody and cellular inducing regimes from experiments 1-4 were compared.

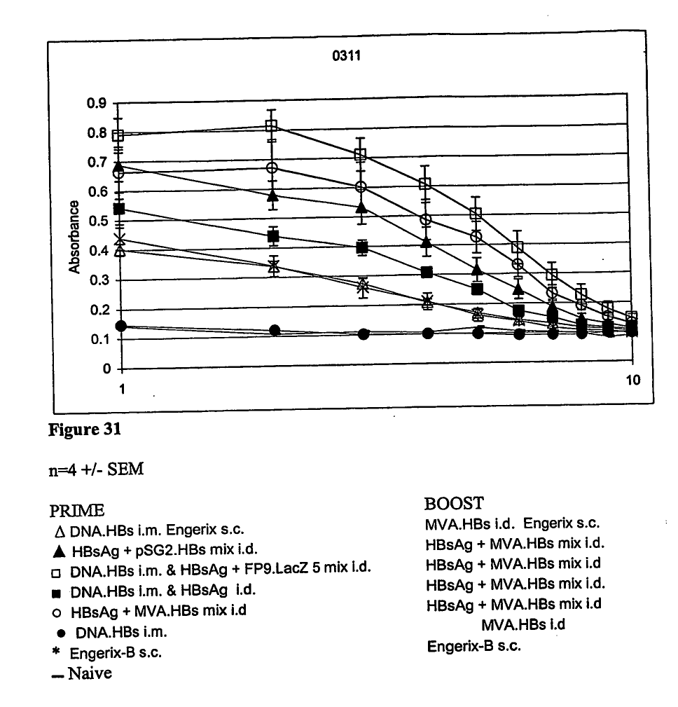

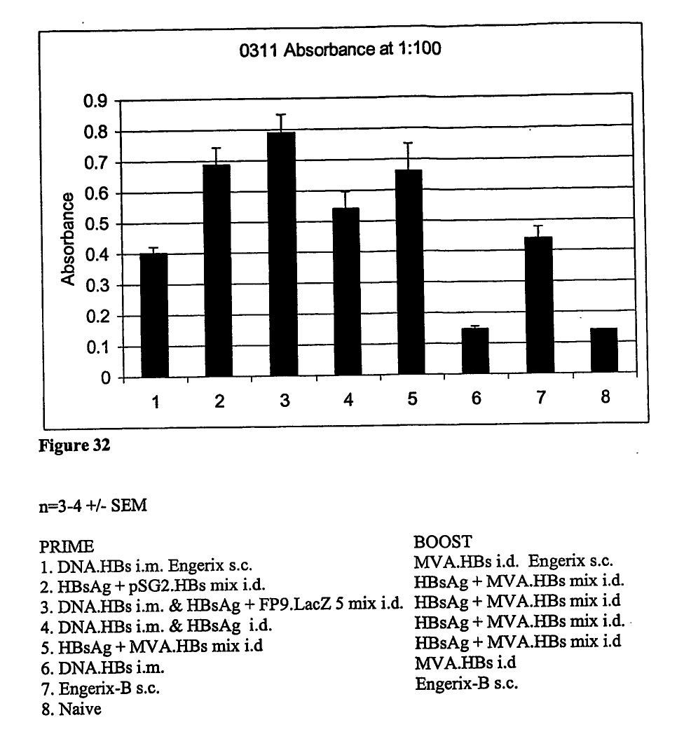

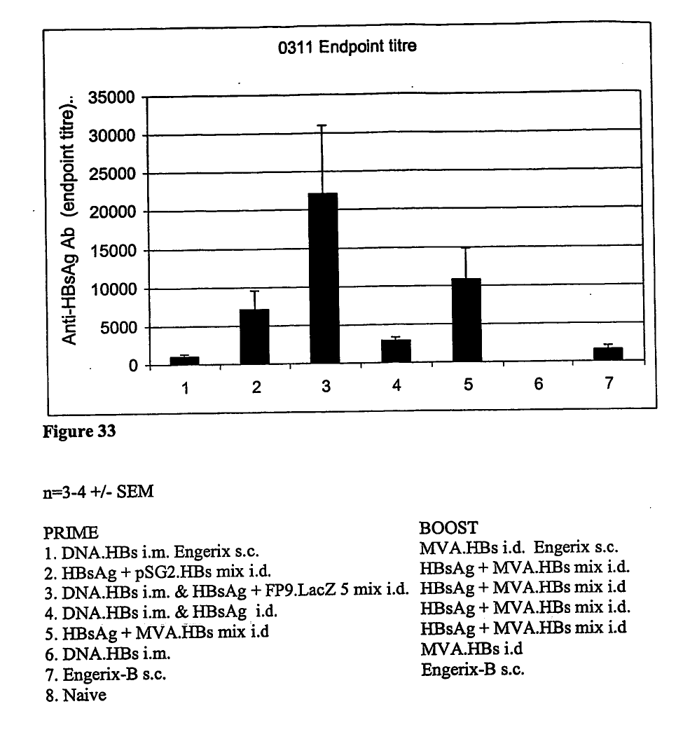

5.1 Antibody Responses (FIGS. 31 to 33)

5.2 T-Cell Responses

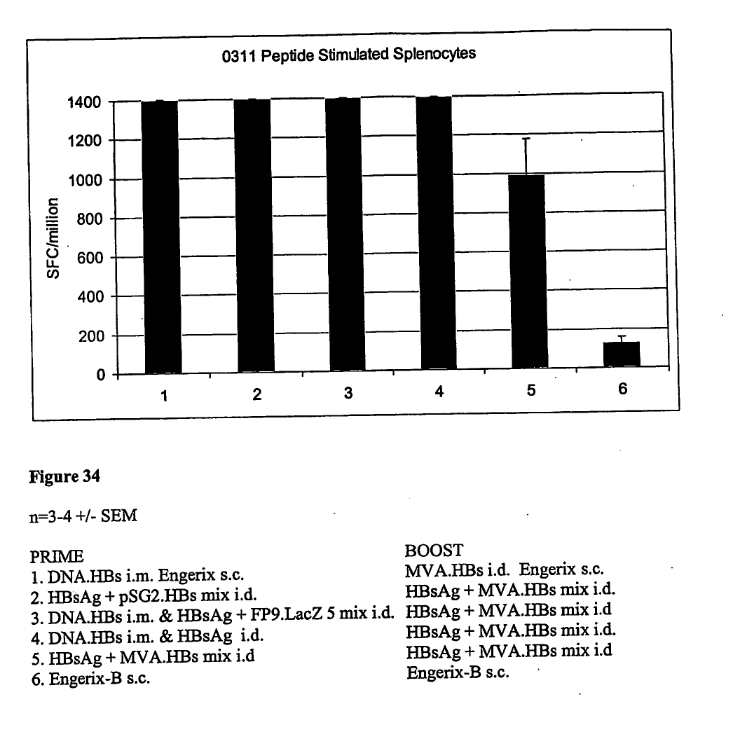

5.2.1 Peptide Stimulated Splenocytes (FIG. 34)

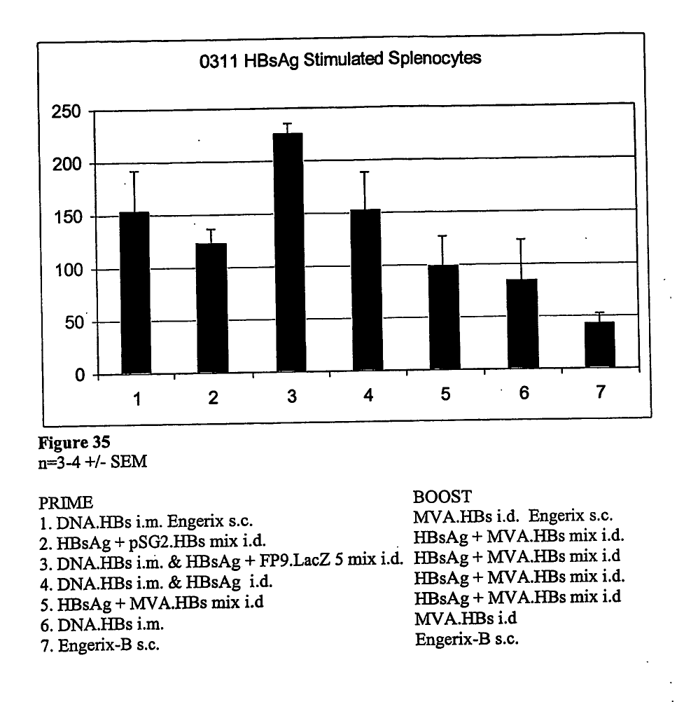

5.2.2 HBsAg Stimulated Splenocytes (FIG. 35)

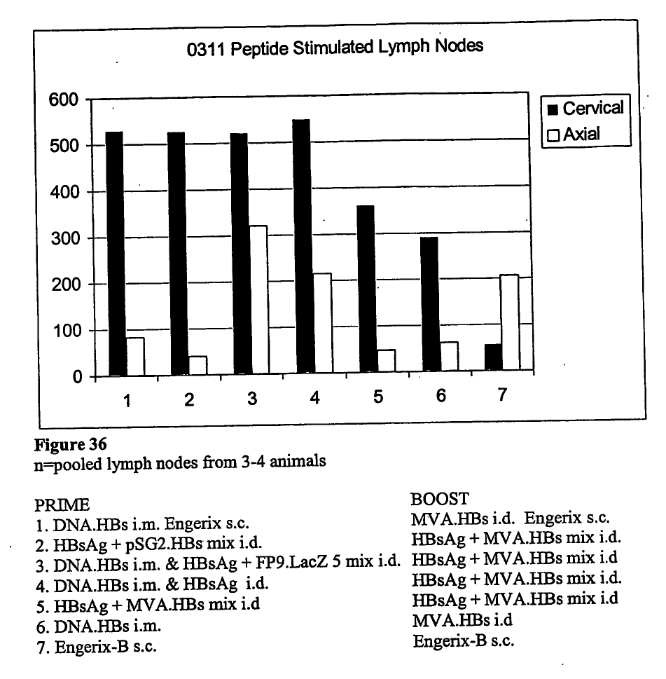

5.2.3 Peptide Stimulated Lymph Nodes FIG. 36)

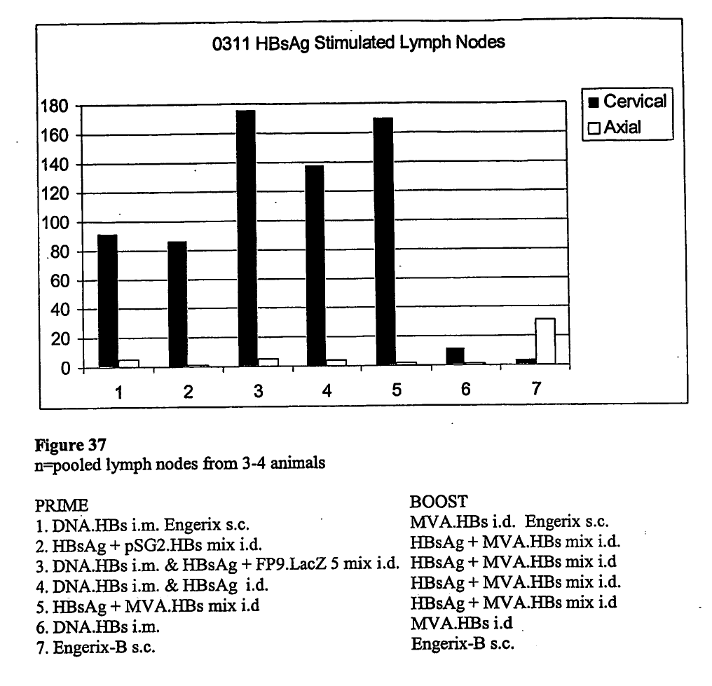

5.2.4 HBsAg Stimulated Lymph Nodes (FIG. 37)

5.3 T-Cell Responses in Blood

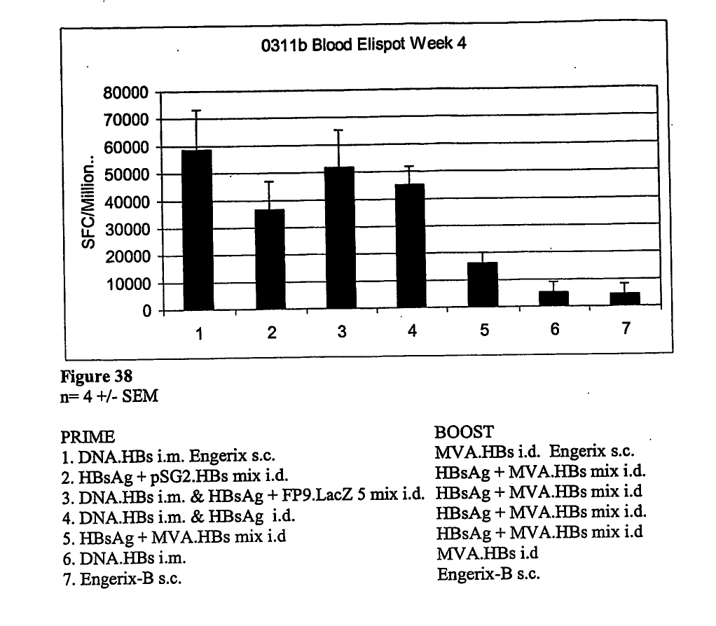

5.3.1 0311b Blood Elispot Week 4 (FIG. 38)

0311b was run concurrently with 0311 and will continue until week 12. Blood is taken for ELISA and blood Elispot weekly until sacrifice at week 12.

### Summary of Responses to Favourable Regimes Across Experiments 2-5

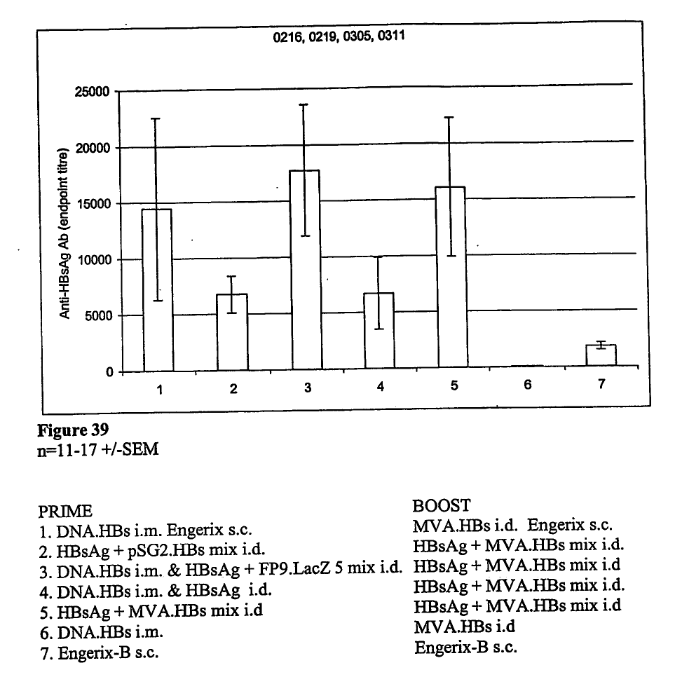

Antibody Responses (FIG. 39)

T-Cell Responses

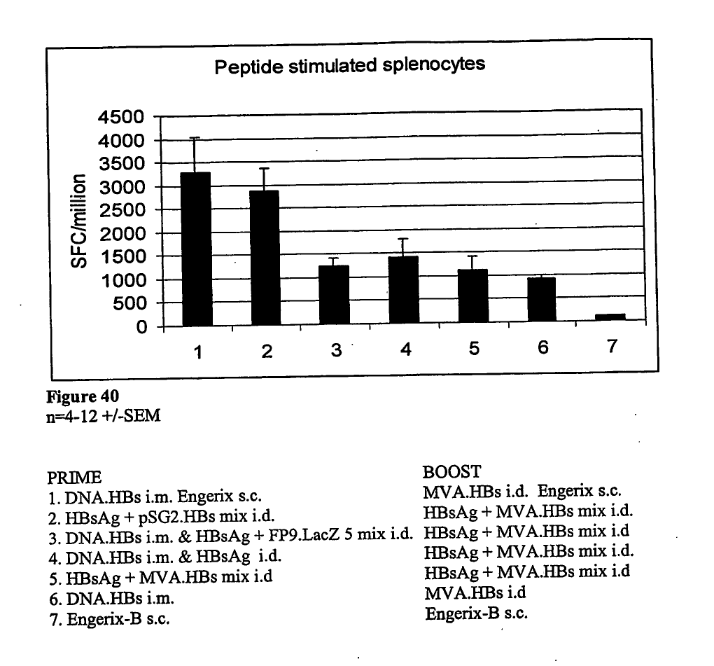

Peptide Stimulated Splenocytes (FIG. 40)

HBsAg Stimulated Splenocytes (FIG. 41)

### Experiment 6

Aim; to establish whether use of Adenovirus vectors boosts antibody and cellular responses to HBsAg. Furthermore, to establish whether heterologous immunisation induces strong cellular and humoral responses without DNA priming.

The CSP (circumsprozoite protein) from Plasmodium berghei (Pb) is used in some groups as the antigen, delivered using an Adenoviral vector.

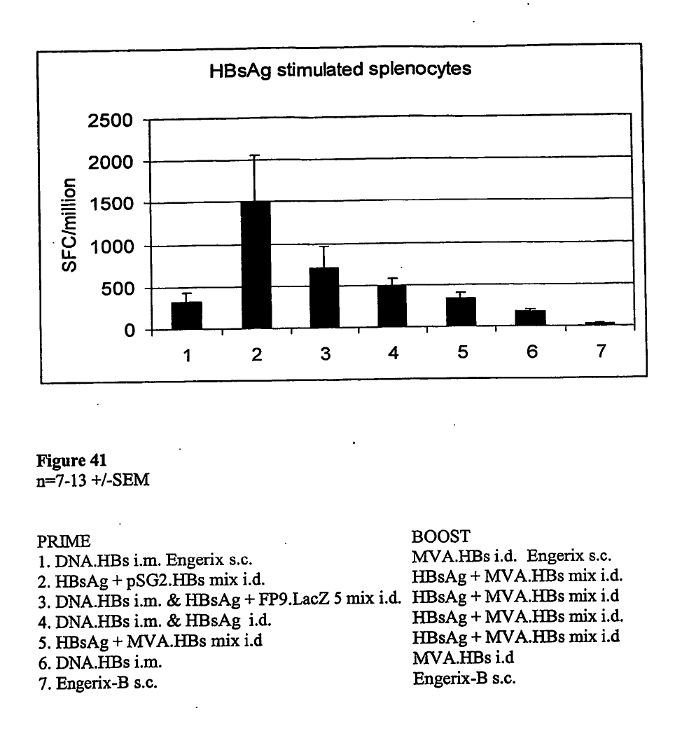

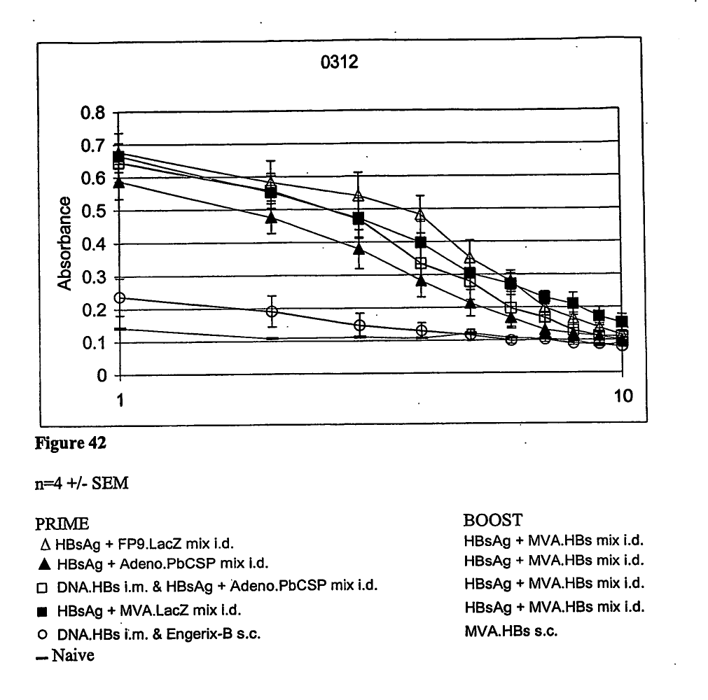

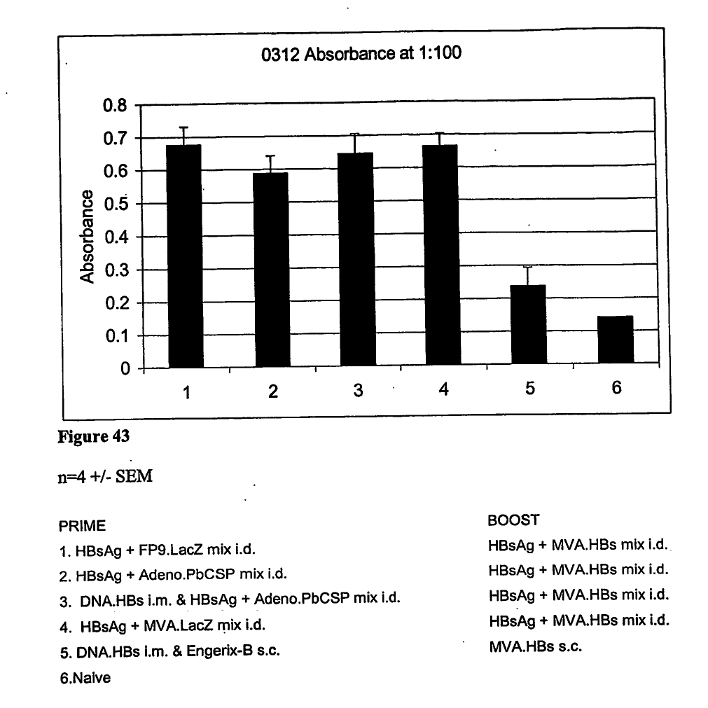

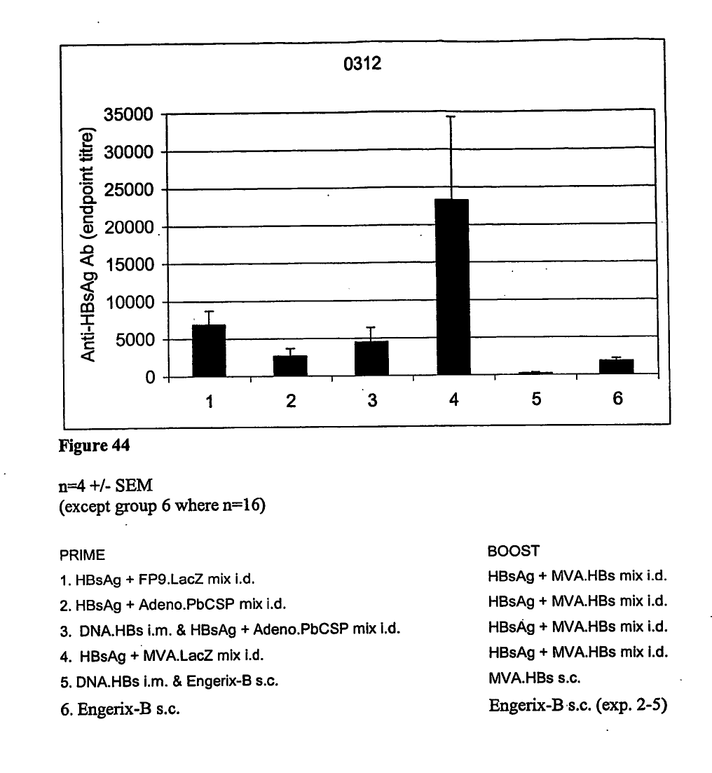

6.1 Antibody Responses (FIGS. 42 to 44)

6.2 T-Cell Responses

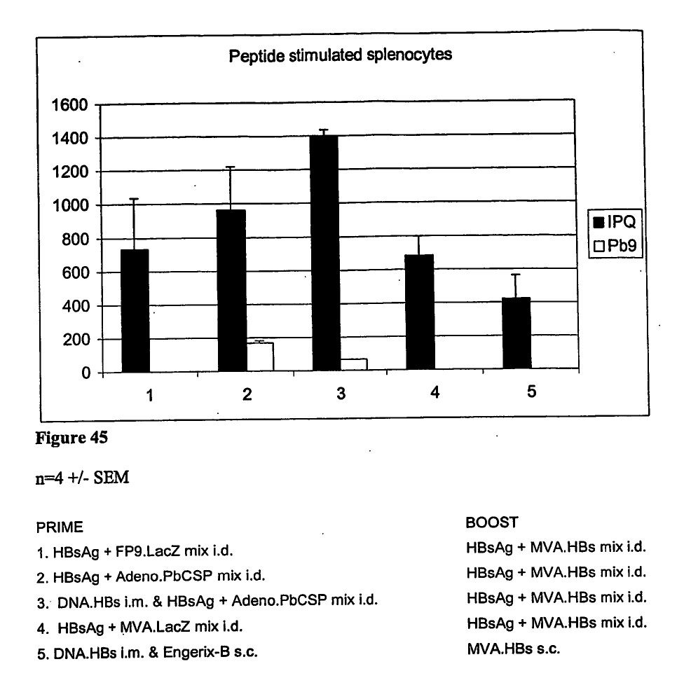

6.2.1 Peptide Stimulated Splenocytes (FIG. 45)

for groups 2 and 3, the CSP epitope was varied between the IPQ epitope and the Pb9 epitope

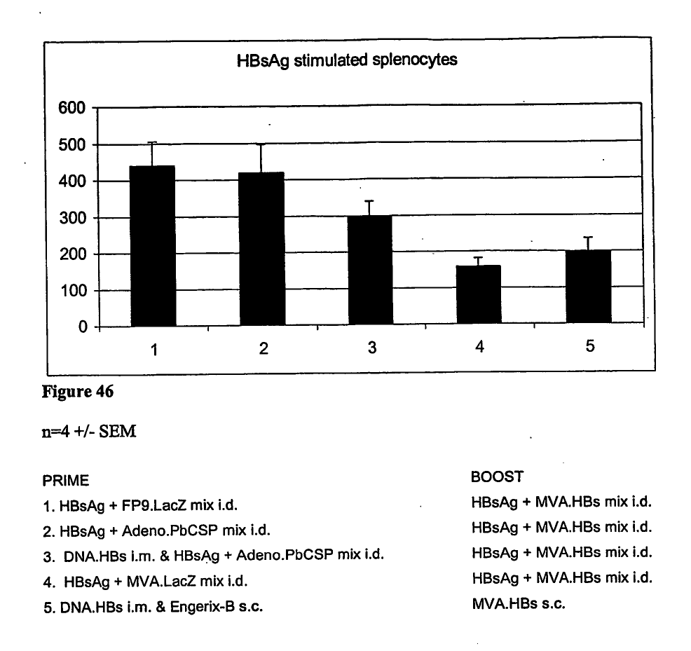

6.2.2 HBsAg Stimulated Splenocytes (FIG. 46)

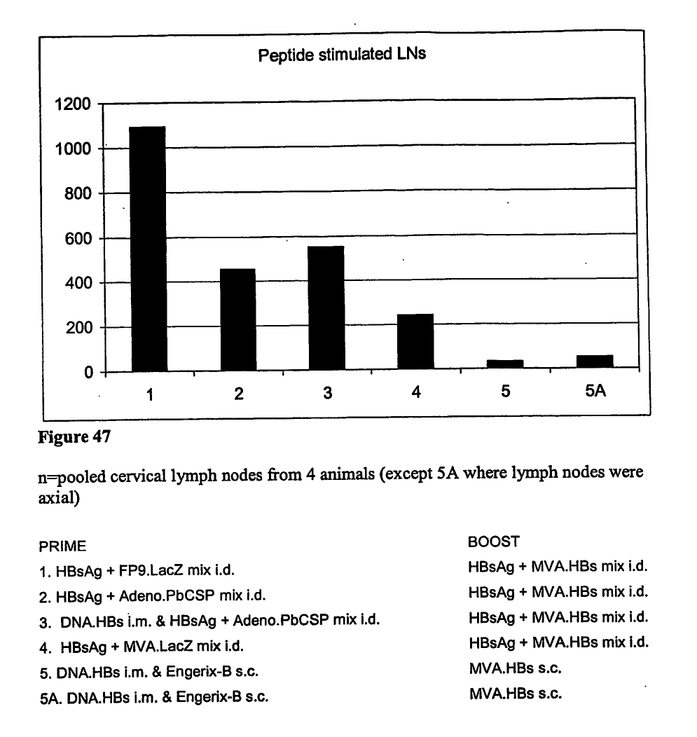

6.2.3 Peptide Stimulated Lymph Nodes (FIG. 47)

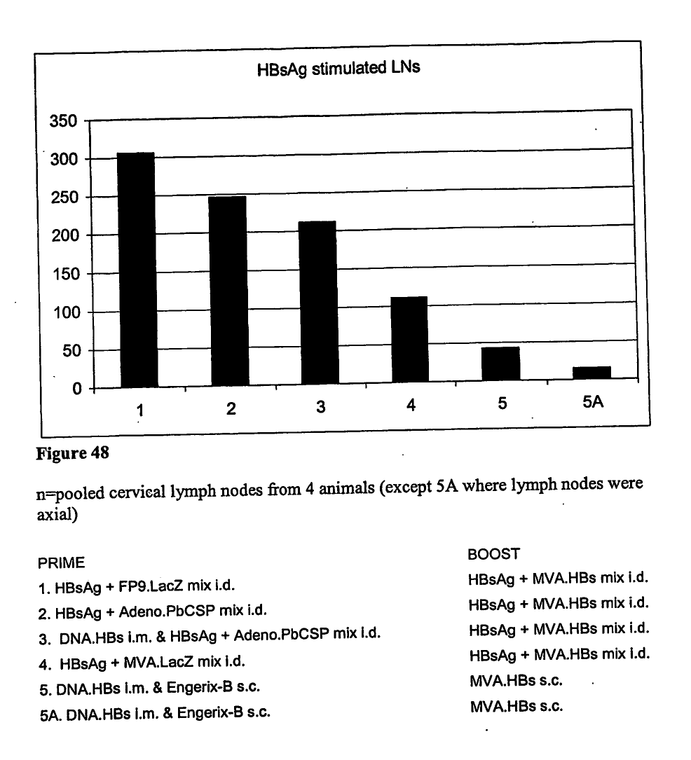

6.2.4 HBsAg Stimulated Lymph Nodes (FIG. 48)

### Experiment 7

Homologous immunization with HBsAg mixed with non-recombinant MVA, NYVAC, ALVAC, FP or ADV. The non-recombinant poxviruses MVA(MVAnr), FP(FPnr), ALVAC(ALVACnr) and NYVAC(NYVACnr) adjuvant potent antibody responses when co-administered i.d. (intradermally) twice with rHBsAg.

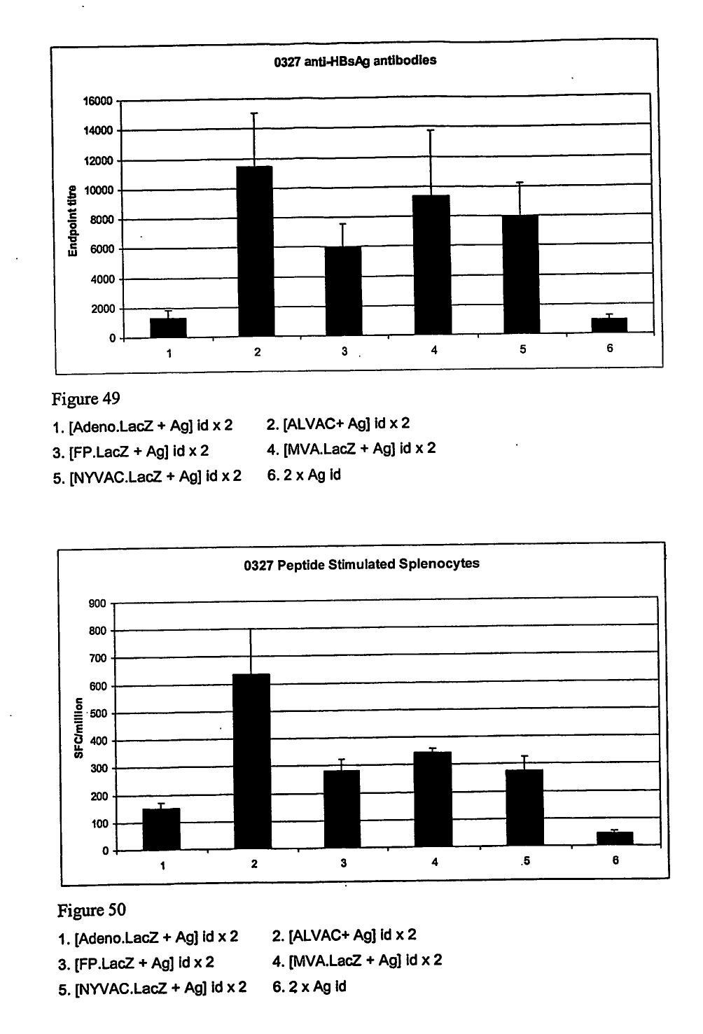

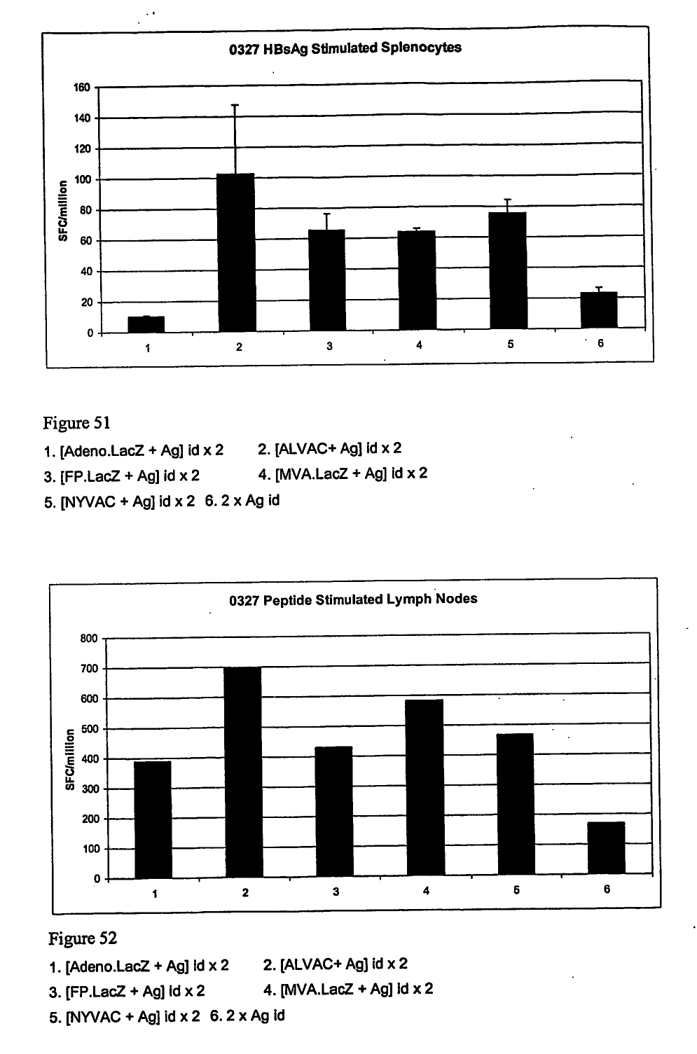

All poxviruses elicited specific antibodies against rHBsAg whereas ADVnr failed to increase antibody responses above the level of repeat rHBsAg immunization (FIG. 49). The attenuated vaccinia virus, NYVACnr, induced similar antibody responses to MVAnr while the avipox virus, ALVACnr elicited the highest levels of anti-rHBsAg antibodies. All viruses increased cellular responses compared to repeat immunization with rHBsAg (FIGS. 50 & 51). The most significant increase in T cell responses compared to rHBsAg immunization were induced by ALVACnr (peptide: p=0.029, rHBsAg: p=0.021). However, non-recombinant MVA, FP, NYVAC and ADV also significantly increased T cell responses compared to rHBsAg immunization (Peptide: p=0.017, 0.021, 0.021, 0.021, rHBsAg: p=0.006, 0.042, 0.021, 0.018). All viruses induced potent responses to peptide in facial DLN (Drainig Lymph Nodes) although, surprisingly, the most potent responses were induced by ALVACnr (FIGS. 52 & 53).

Homologous immunization with non-recombinant poxvirus mixed with antigen is an efficient method for inducing potent antibody and cellular responses against the co-administered protein. It can be seen here that although adenovirus is effective, poxvirus is preferred.

Co-administration of a non-recombinant or “empty” viral vector, that does not encode an antigen, together with an antigen induces increased antibody and T cell responses compared to the antigen alone. In particular, it can be seen that a homologous prime boost regimen is preferable and, furthermore, that ALVAC is particularly effective as the vector.

### Experiment 8 Longevity of Favourable Regimes in 0311b

Favourable antibody and cellular inducing regimes from experiments 1-4 were analysed from day 0 (day of prime) until day 148 for induction of antibodies to HBsAg and day 216 for cellular response to peptide.

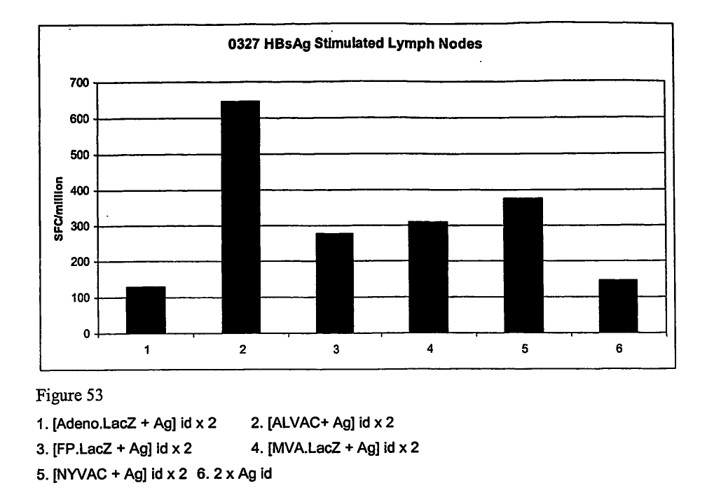

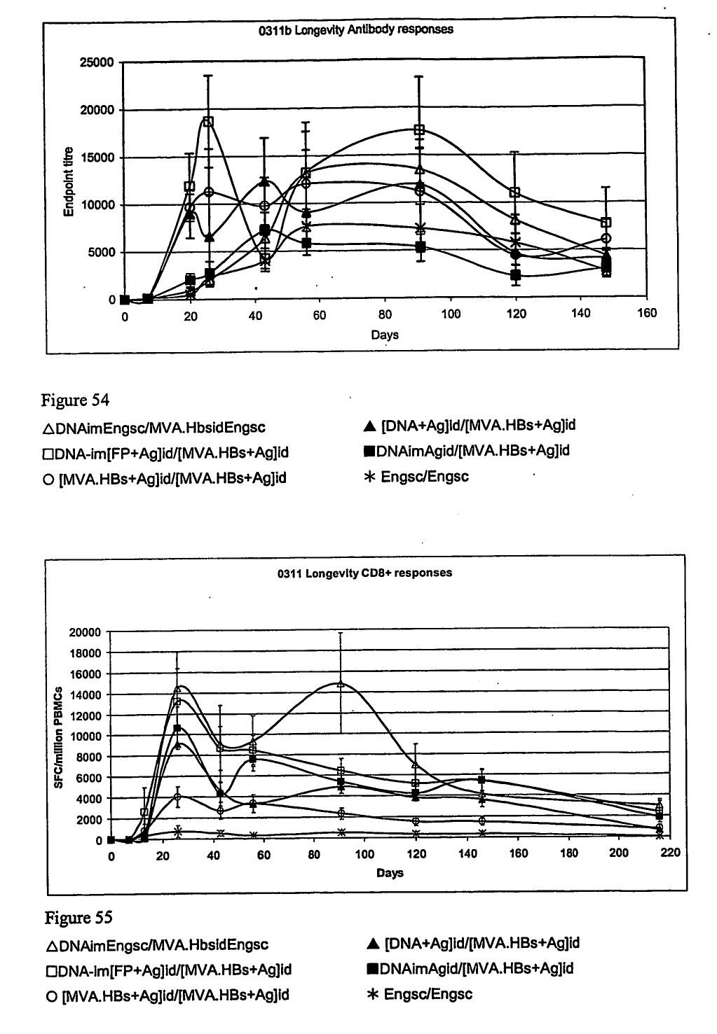

Immunisation with DNA i.m. [FP+Ag] i.d./[MVA.HBs+Ag]i.d. induced the most potent and long-lived responses to HBsAg although all antibody levels in all other regimens except DNAi.m.Ag i.d./[MVA.HBs+Ag]i.d. were consistently higher than repeat Engerix-B immunization until day 120 when all responses were in decline (FIG. 54).

All regimens induced much greater CD8⁺ T cell responses to peptide when compared to repeat Engerix-B immunization (FIG. 55). Immunisation with DNA i.m. Engerix-B s.c./MVA.Hbs i.d. Engerix-B s.c. induced higher T cell responses to peptide than other regimens until day 120 post-prime. These results demonstrate the longevity of responses induced by the combination of T cell and antibody inducing vaccines.

### Experiment 9 Further ELISA data for Experiments 1-7

Further ELISA's were carried out on combinations taken from Experiment 1-7. The results are shown in FIG. 56.

### Experiment 10 Viral Modulation of IgG Subclasses

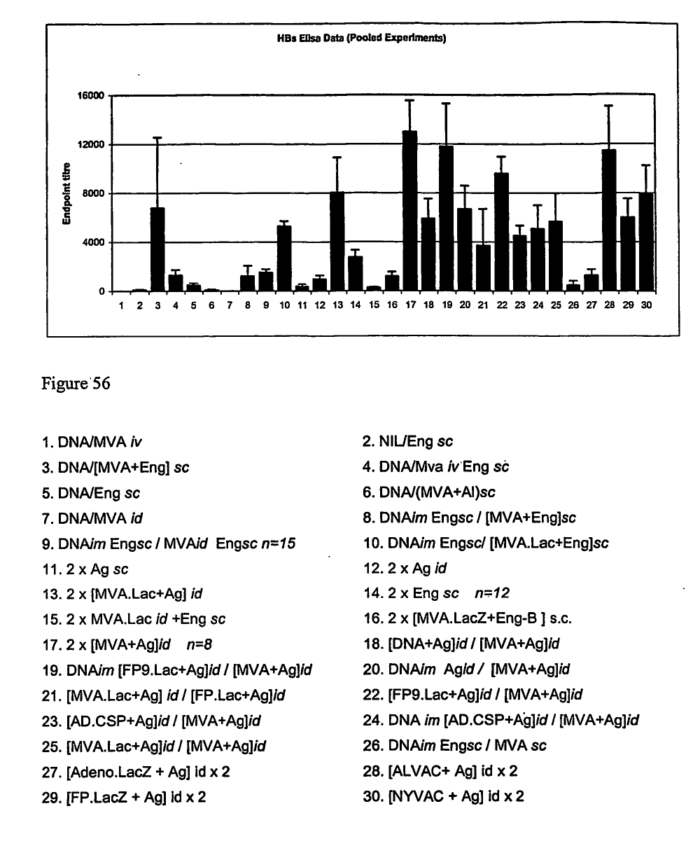

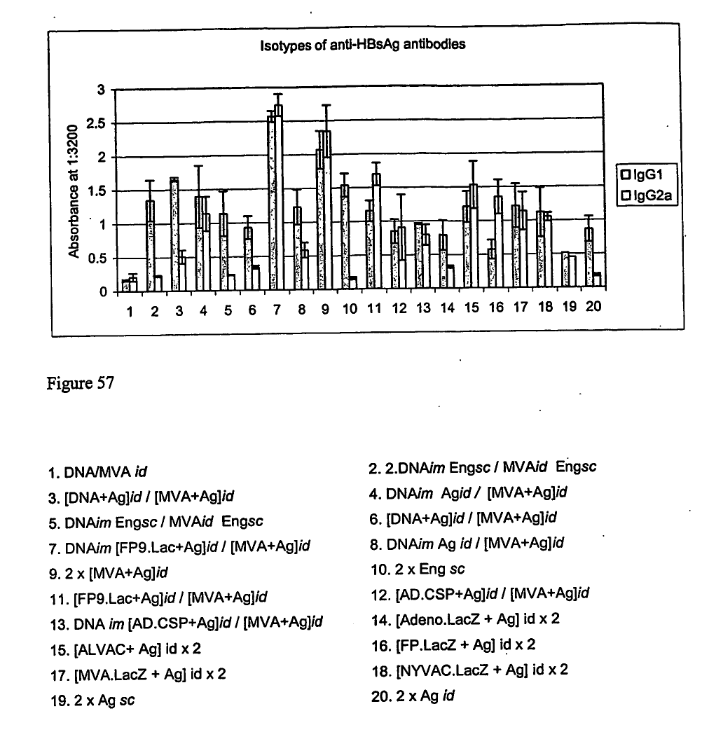

The ratio of isotype subclasses IgG1 and IgG2a gives an indication of Th2 or Th1 bias of humoral responses respectively. The effect of DNA, poxviruses and ADV on IgG subclass division was determined (FIG. 57) by measuring IgG subclasses/isotypes using biotin conjugated anti-mouse IgG1 or IgG2a antibody (Pharmingen) followed by incubation with ExtrAvidin (Sigma) for isotype analysis.

Engerix-B predominantly induced IgG1 antibodies which is indicative of a Th2 biased humoral response. This concurs with the poor IFN-γ production by peptide and rHBsAg stimulated splenocytes from Engerix-B immunized animals (FIG. 15, group 4 and FIG. 16, group 4).

Priming with a mixture of rHBsAg and recombinant MVA or non-recombinant FP, MVA, ALVAC, NYVAC with or without DNA induced equal levels of IgG1/IgG2a or higher IgG2a. Surprisingly, homologous immunization with rHBsAg and the avipox viruses ALVACnr and FPnr consistently induced a higher level of IgG2a than IgG1. In contrast, combination of rHBsAg with non-recombinant MVA and NYVAC, both derived from vaccinia, induced a 1:1 ratio of IgG1:IgG2a. Absence of poxvirus in the prime predominantly lead to increased levels of IgG1 compared to IgG2a except in DNA/MVA where low levels of both IgG1:IgG2a were observed.

Conversely, co-administration of ADVnr induced higher IgG1 than IgG2a similar to responses following repeat immunization with rHBsAg alone, indicating a lack of adjuvant activity or antibody response of ADVnr.

The increased levels of IgG2a induced by poxviral priming indicates a Th1 biased humoral response, while priming with combinations of DNA, Engerix-B and rHBsAg lead to a higher ratio of IgG1 and therefore Th2 bias. Recombinant MVA, MVAnr and NYVACnr induced an even ratio of IgG1:IgG2a whereas FP and ALVAC skew the response to IgG2a . These results indicated that equal Th-1 and Th-2 or biased responses could be primed to the same antigen depending on the co-delivered viral vector.

### Experiment 11:P berghei Murine Model

This investigated the P berghei murine model of malaria. As with the Hepatitis B model in previous experiments, this experiment combines a T-cell inducing vaccine with an antibody inducing vaccine leading to potent induction of both responses concurrently.

Mice were primed at day 0 and boosted on day 22. T cell responses to a dominant CD8⁺ epitope of CSP (Pb9) were measured in blood on day 21 and 36. Antibody responses to the B-cell epitope (DP₄NPN) present in both the Alum-precipitated vaccine (Apv) and CSP encoded by DNA, MVA and FP as well as HBc responses were measured on days 14 and 37. The terms AP and Apv are used interchangeably.

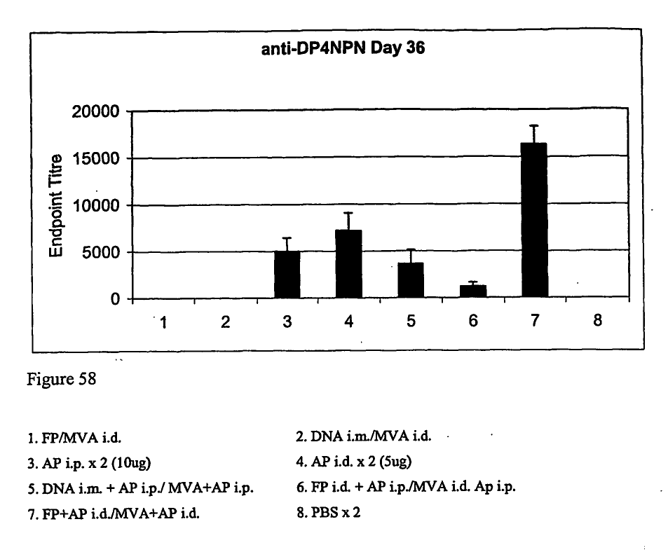

No significant levels of antibodies to DP₄NPN or HBc in any vaccine regimen were detected at day 14. The vaccine regimens of PBS×2, DNA i.m./MVA i.d. and MVA i.d./FP i.d. also failed to induce detectable levels of anti-DP₄NPN antibodies on day 37 (FIG. 58). Repeat immunization with 5 μg of Apv i.d. and 10 μg of Apv i.p. strongly induced antibodies to DP₄NPN although i.d. immunization induced slightly higher responses. Conversely, the immunization regimens of DNA i.m.+AP i.p./MVA+AP i.p. and FP i.d.+AP i.p./MVA i.d. Ap i.p. reduced antibody levels compared to repeat Apv immunization. However the regimen FP+AP i.d./MVA+AP i.d., where FP and MVA were mixed with Apv and administered i.d., greatly enhanced antibody responses to DP₄NPN compared to repeat Apv immunization.

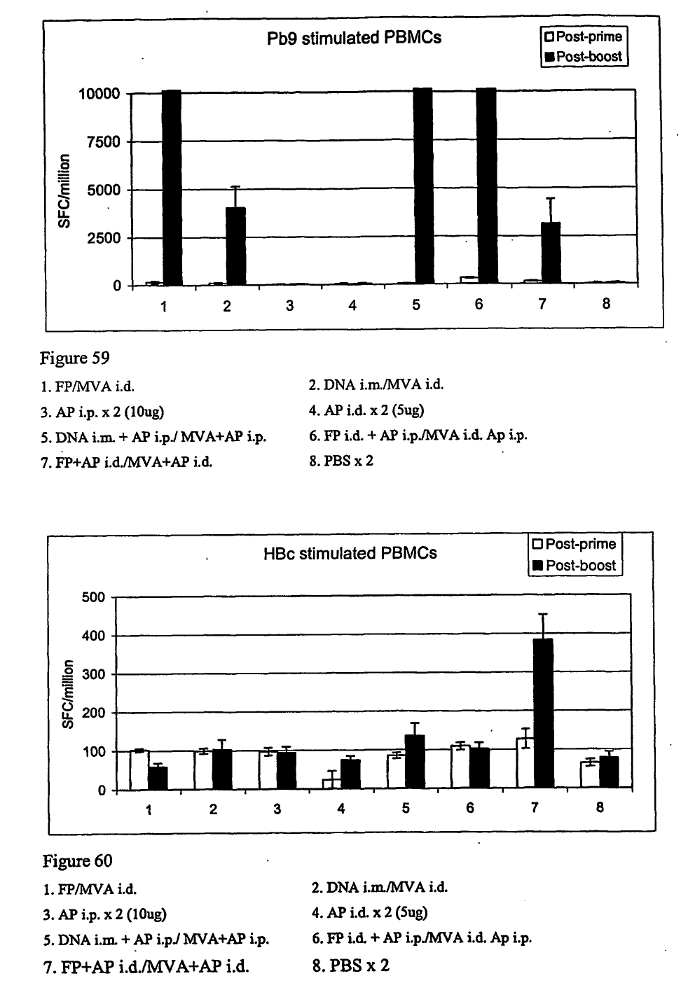

Immunization with DNA i.m./MVA i.d. and FP i.d./MVA i.d. both induce high levels of peptide specific T cells although PP/MVA induced a greater level than DNA/MVA (FIG. 59). Repeat immunisation with Apv i.d. or i.p. failed to induce any T cell response to peptide above background. Combination of DNA/MVA with Apv (DNA i.m.+AP i.p./MVA+AP i.p.) lead to a great increase in T cell responses to peptide compared to DNA/MVA immunisation. Concurrent immunization with FP/MVA and Apv at separate sites (FP i.d.+AP i.p./MVA i.d. Ap i.p.) induced similar levels of peptide specific T cells to FP/MVA immunization. However, if Apv was mixed with FP/MVA and administered i.d. (FP+AP i.d./MVA+AP i.d.), T cell responses to peptide were reduced when compared to immunization with FP/MVA alone.

Stimulation of PBMCs with HBc induced minimal responses in all regimens except FP+AP i.d./MVA+AP i.d. where these responses were clearly amplified (FIG. 60). This increase in T cells specific for HBc may represent both CD4⁺ and CD8⁺ populations and are an example of the capability of FP and MVA to adjuvant a protein that is not encoded within either virus. It also illustrates the virus' ability to adjuvant T cell responses in the presence of alum. This is surprising, as the presence of alum would have been expected to negate or at least counteract any T cell response.

### Experiments 12 and 13

Experiments 12 and 13 provide further evidence that the use of a vector in combination with an antigen enhances the level of protection provided, compared to the use of the antigen alone.

### Experiment 12

1. Schedule

The schedule of immunisations used is shown in table 1, below.

Vaccines:

FP9.PbCSP: Fowlpox expressing full length P. berghei CSP
MVA.PbCSP: Modified Virus Ankara expressing full length P. berghei CSP
FP9.LacZ: Fowlpox encoding the marker β-galactosidase
MVA.LacZ: Modified Virus Ankara encoding the marker β-galactosidase
Apovia: Hepatitis B core(HBc) particles containing two copies of the P. berghei CS
repeat (B-cell epitopes only) between amino acids D78 and P79 of HBc with or without Alum.

PBS: Phosphate buffered saline, sterile and endotoxin free.

TABLE 1
CN0401 6 mice/group

| | | | | Challenge | | AIM |
|---|---|---|---|---|---|---|
| | | | Serum | 1000 | | Concurrent |
| | | | for | sporozoites | | generation of |
| | Prime - Day 0 | | ELISA | i.v. | | strong T cell |
| | 25th February | Boost - Day 14 | and | 24th March | Mouse | and humoral |
| Group | 2004 | 11th March 2004 | ELIspot | 2004 Day 28 | number | immunity |
| 1 | 1 × 10⁶ pfu | 1 × 10⁶ pfu | | 66.6% | 1-6 | What is the |
| | FP9.PbCSP | MVA.PbCSP | | Protection (4 | | effect of |
| | admixed with | admixed with 5 μg | | out of 6) | | removing alum |
| | 5 μg Apovia | Apovia adsorbed to | | | | from the |
| | adsorbed to alum | alum i.d. | | | | Apovia |
| | i.d. | | | | | Vaccine? |
| 2 | 1 × 10⁶ pfu | 1 × 10⁶ pfu | 22nd | 100% | 7-12 | Is recombinant |
| | FP9.PbCSP | MVA.PbCSP | March | Protection | | FP and MVA |
| | admixed with | admixed with 5 μg | 2003 Day | (6 out of 6) | | (and therefore |
| | 5 μg Apovia (no | Apovia (no alum) | 26 | | | specific T cells) |
| | alum) i.d. | i.d. | | | | required for |
| | | | | | | protection? |
| 3 | 1 × 10⁶ pfu | 1 × 10⁶ pfu | | 50% | 13-18 | |
| | FP9.LacZ | MVA.LacZ | | Protection (3 | | |
| | admixed with | admixed with 5 μg | | out of 6) | | |
| | 5 μg Apovia | Apovia adsorbed to | | | | |
| | adsorbed to alum | alum i.d. | | | | |
| | i.d. | | | | | |
| 4 | 1 × 10⁶ pfu | 1 × 10⁶ pfu | | 50% | 19-24 | |
| | FP9.LacZ | MVA.LacZ | | Protection (3 | | |
| | admixed with | admixed with 5 μg | | out of 6) | | |
| | 5 μg Apovia (no | Apovia (no alum) | | | | |
| | alum) i.d. | i.d. | | | | |
| 5 | 1 × 10⁶ pfu | 1 × 10⁶ pfu | | 66.6% | 25-30 | |
| | FP9.PbCSP i.d. | MVA.PbCSP i.d. | | Protection (4 | | |
| | | | | out of 6) | | |
| 6 | 5 μg Apovia | 5 μg Apovia | | No | 31-36 | |
| | adsorbed to alum | adsorbed to alum | | Protection | | |
| | i.d. | i.d. | | | | |
| 7 | 5 μg Apovia (no | 5 μg Apovia (no | | 16.6% | 37-42 | |
| | alum) i.d. | alum) i.d. | | Protection (1 | | |
| | | | | out of 6) | | |
| 8 | PBS i.d. | PBS i.d. | | No | 43-48 | |
| | | | | Protection | | |

2. Blood Elispot Results

PBMCs collected from individual animals were stimulated with the P. berghei CD8+ peptide Pb9(SYIPSAEKI) and 1123 (whole HBC particle) for 18-22 hours. Bars represent the mean IFN-gamma spot forming cells for six animals +/−SEM.

2.1 Pb9 Stimulated PBMCs.

The results are shown in FIG. 61.

All CD8+T cell responses were minimal on day 21 post-prime (prior to boosting, data not shown).
T cell responses were increased above FP.CSP/MVA.CSP immunisation when FP.CSP and MVA.CSP were combined with un-adjuvanted apovia vaccine
   2.2 1123 Stimulated PBMCs.

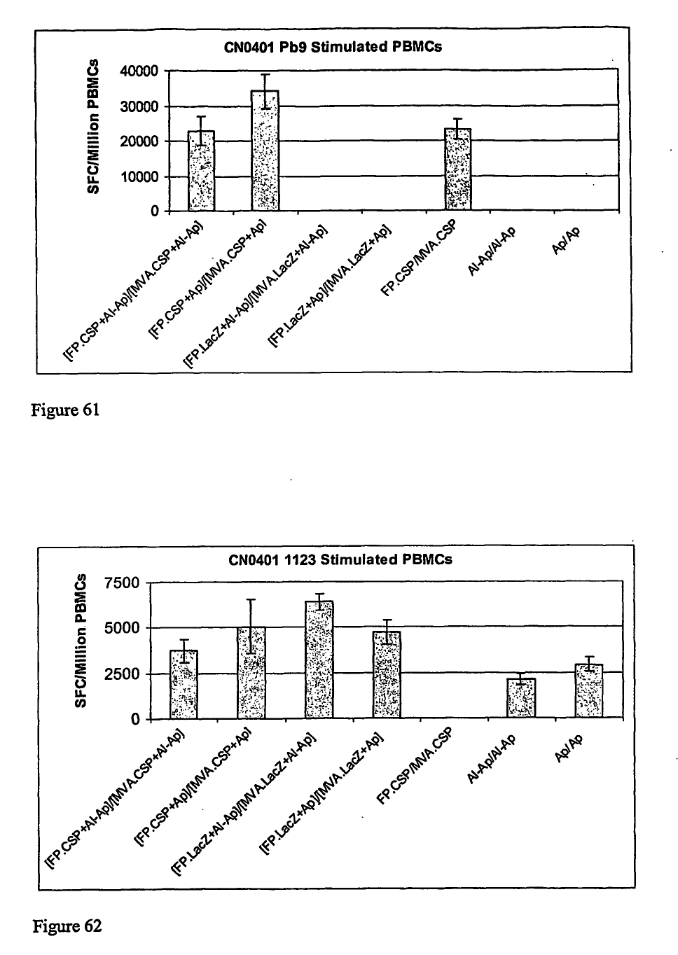

The results are shown in FIG. 62.
All viruses co-administered with Apovia vaccine enhanced T cell responses to the Apovia carrier protein 1123 when compared to immunization with Apovia vaccine alone.
   3. Serum ELISA Results

Serum collected from individual animals were tested for antibodies against (DP4NPN)2 (P. berghei CS repeats) and 1123 (whole HBC particle) Bars represent the mean endpoint titre for six animals ±SEM.

3.1 Anti(DP4NPN)2 Antibodies

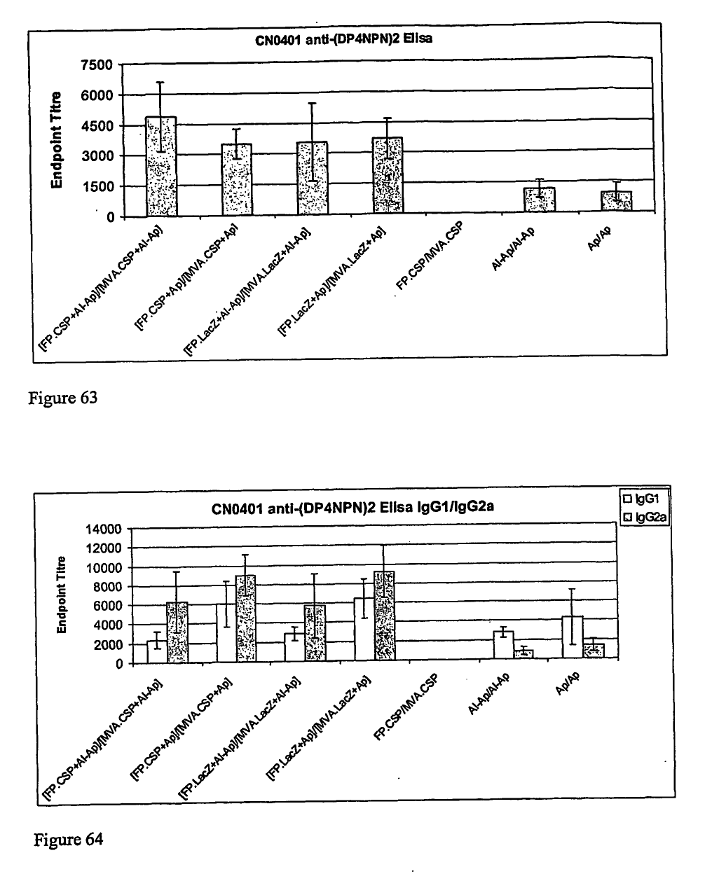

The results are shown in FIG. 63.
Combination vaccines induced the highest antibody levels when compared to immunization with Apovia vaccine with or without alum
   3.2 Isotype Subclass of Anti-(DP4NPN)2 Antibodies

The results are shown in FIG. 64.
All combination vaccines induced potent IgG2a responses compared to Apovia immunisation.
Combination vaccines without alum also increased IgG1 responses compared to Apovia immunization.
   4. Conclusions

Group 1: Prime FP9.PbCSP+Apovia adsorbed to alum i.d.

Boost: MVA.PbCSP+Apovia adsorbed to alum i.d.

Group 2: Prime: FP9.PbCSP+Apovia (no alum) i.d.

Boost: MVA.PbCSP+Apovia (no alum) i.d.

Group 3: Prime: FP9.LacZ+Apovia adsorbed to alum i.d.

Boost: MVA.LacZ+Apovia adsorbed to alum i.d.

Group 4: Prime: FP9.LacZ+Apovia (no alum) i.d.

Boost: MVA.LacZ+Apovia (no alum) i.d.

Group5: Prime: FP9.PbCSP i.d.

Boost: MVA.PbCSP i.d.

Group 6: Prime: Apovia adsorbed to alum i.d.

Boost: Apovia adsorbed to alum i.d.

Group 7: Prime: Apovia (no alum) i.d.

Boost: Apovia (no alum) i.d.

Group 8: Prime: PBS i.d.

Boost: PBS i.d.

Short-Term Challenge Conclusions
Apovia adsorbed to alum mixed with FP.CSP and MVA.CSP did not increase rotection above immunization with FP.CSP and MVA.CSP (1 vs 5).
Apovia without alum mixed with FP.CSP and MVA.CSP lead to 100% protection (Group 2). This response is synergistic rather than additive (group 2 vs 5+7).
Non-recombinant FP and MVA adjuvanted Apovia, with or without alum (group 2 and 3) and also enhanced protection (groups 2+3 vs 6+7).
Regimens that induced antibodies and T cells concurrently showed an isotype pattern that was skewed towards IgG2a.
Group 2

### Experiment 13

1. Schedule

TABLE 2
0326 6 mice/group

| | | | | Challenge | | |
|---|---|---|---|---|---|---|
| | | | | 1000 | | AIM |
| | | | Serum | sporozoites | | Concurrent |
| | | | for | i.v. | | generation of |
| | Prime - Day 0 | | ELISA | 6th | | strong T cell and |
| | 12th November | Boost - Day 22 | and | February | Mouse | humoral |
| Group | 2003 | 4th December 2003 | ELIspot | 2004 Day 86 | number | immunity |
| 1 | 1 × 10⁶ pfu | 1 × 10⁶ pfu | 3rd | No | 1-6 | |
| | FP9.PbCSP i.d. | MVA.PbCSP i.d. | December | Protection | | |
| | | | 2003 Day | | | |
| | | | 21 | | | |
| 2 | 50 μg | 1 × 10⁶ pfu | 18th | No | 7-12 | |
| | DNA.PbCSP i.m. | MVA.PbCSP i.d. | December | Protection | | |
| | | | 2003 Day | | | |
| | | | 36 | | | |
| 3 | 10 μg Apovia | 10 μg Apovia | 20th | 33% | 13-18 | |
| | adsorbed to alum | adsorbed to alum i.p. | January | protection (2 | | |
| | i.p. | | 2004 Day | out of 6) | | |
| | | | 69 | | | |
| 4 | 5 μg Apovia | 5 μg Apovia | 2nd | No | 19-24 | |
| | adsorbed to alum | adsorbed to alum i.d. | February | Protection | | |
| | i.d. | | 2004 Day | | | |
| | | | 82 | | | |
| 5 | 50 μg DNA.PbCSP | 1 × 10⁶ pfu | Post | 50% | 25-30 | |
| | i.m. and 10 μg | MVA.PbCSP | challenge | protection (3 | | |
| | Apovia adsorbed | admixed with 10 μg | protected: | out of 6) | | |
| | to alum i.p | Apovia adsorbed to | 25^{th} | | | |
| | | alum i.p. | February | | | |
| | | | 2004 Day | | | |
| | | | 105 | | | |
| 6 | 1 × 10⁶ pfu | 1 × 10⁶ pfu | | 66% | 31-36 | |
| | FP9.PbCSP i.d. | MVA.PbCSP i.d. and | | protection (4 | | |
| | and 10 μg Apovia | 10 μg Apovia | | out of 6) | | |
| | adsorbed to alum | adsorbed to alum i.p. | | | | |
| | i.p. | | | | | |
| 7 | 1 × 10⁶ pfu | 1 × 10⁶ pfu | | 100% | 37-42 | Mouse 38 died |
| | FP9.PbCSP | MVA.PbCSP | | protection (5 | | post-boost |
| | admixed with 5 μg | admixed with 5 μg | | out of 5) | | |
| | Apovia adsorbed | Apovia adsorbed to | | | | |
| | to alum i.d. | alum i.d. | | | | |
| 8 | PBS i.d. | PBS i.d. | | No | 43-48 | |
| | | | | Protection | | |

The schedule of immunisations used is shown in table 2,above.

Vaccines:
FP9.PbCSP: Fowlpox expressing full length P. berghei CSP
MVA.PbCSP: Modified Virus Ankara expressing full length P. berghei CSP
DNA.PbCSP: Plasmid DNA expressing full length P. berghei CSP
Apovia: Hepatitis B core(HBc) particles containing two copies of the P. berghei CS repeat (B-cell epitopes only) between amino acids D78 and P79 of HBc.
PBS: Phosphate buffered saline, sterile and endotoxin free.
   2. Blood Elispot Results

PBMCs collected from individual animals were stimulated with the P. berghei CD8+ peptide Pb9(SYIPSAEKI) and 1123 (whole HBC particle) for 18-22 hours. Bars represent the mean IFN-gamma spot forming cells for six animals (five animals only in group 7) +/−SEM.

2.1 Pb9 Stimulated PBMCs

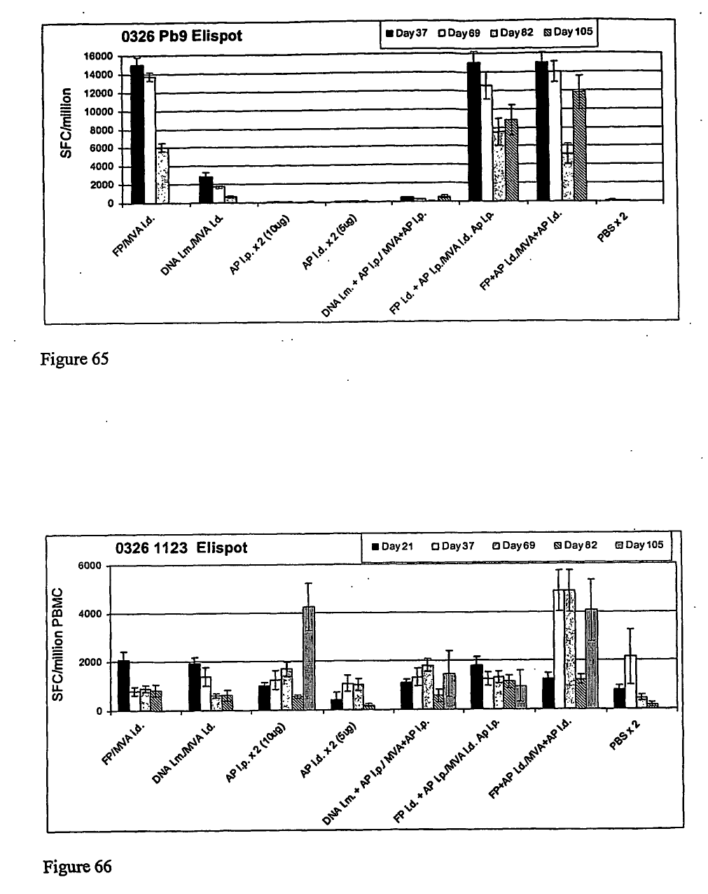

The results are shown in FIG. 65.
All CD8+T cell responses were minimal on day 21 post-prime (prior to boosting, data not shown).
Intra-dermal (i.d.) FP.PbCSP prime and MVA.PbCSP boost with or without Apovia vaccine induced strong T cell responses.
Intra-muscular (i.m.) DNA.PbCSP priming and i.d. MVA.PbCSP boosting induced low CD8+ T cell response which declined further by day 82.
As expected the Apovia vaccine administered twice intra-peritoneally (i.p.) or i.d. induced minimal T cell responses.
Priming with Apovia vaccine i.p. and DNA.PbCSP i.d. followed by i.p. boosting with MVA.PbCSP mixed with Apovia vaccine also induced minimal CD8+ T cell responses.
   2.2 1123 Stimulated PBMCs

The results are shown in FIG. 66.
1123 induced high background levels of IFN-gamma SFC (Approximately 800-2000 SFC) in all groups.
   3. Serum ELISA Results

Serum collected from individual animals were tested for antibodies against (DP4NPN)2 (P. berghei CS repeats) and 1123 (whole HBC particle) Bars represent the mean endpoint titre for six animals (five animals only in group 7) +/−SEM.

3.1 Anti-(DP4NPN)2 Antibodies

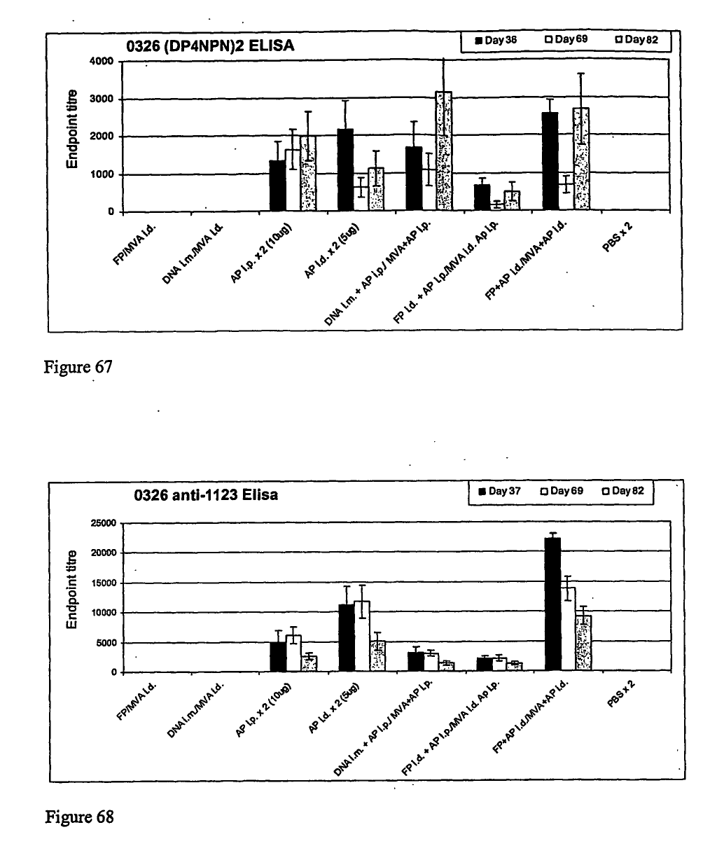

The results are shown in FIG. 67.
No specific antibodies to (DP4NPN)2 were detected following priming with FP.PbCSP i.d. or DNA.PbCSP i.m. and boosting with MVA.PbCSP i.d.
All groups that received Apovia vaccine induced specific antibodies although levels were low in animals primed with DNA.PbCSP i.m. and Apovia vaccine i.p. and boosted with MVA.PbCSP mixed with Apovia vaccine.
In four groups antibody levels dropped down on day 69 and increased again by day 82.
Post-challenge ELISA of sera from surviving animals is pending.
   3.2 Anti-1123 Antibodies

The results are shown in FIG. 68.
As expected, groups that received formulated Apovia vaccine induced specific anti-1123 antibodies.
Responses steadily declined between day 37 and Day 82.
   3.3 Isotype Subclass of Anti-(DP4NPN)2 Antibodies

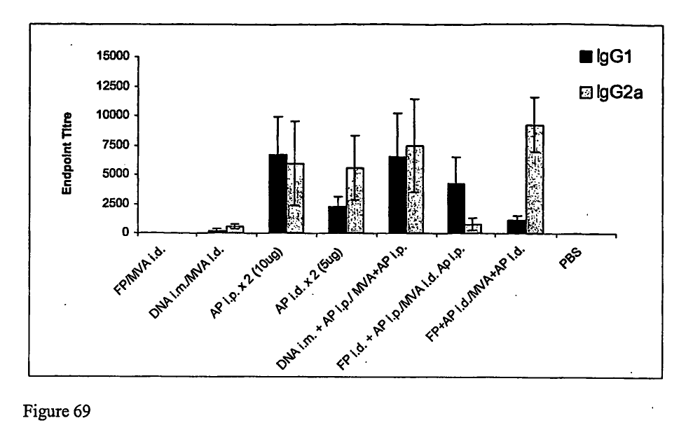

The results are shown in FIG. 69.
Ratios of IgG1 to IgG2a were skewed towards IgG1 when FP.PbCSP and MVA.PbCSP were given i.d. at the same time as Apovia vaccine was administered i.p.
This pattern was reversed if Apovia vaccine was mixed with FP.PbCSP and MVA.PbCSP and co-administered i.d. at prime and boost respectively.
   4. Conclusions

Group 1: Prime: FP9.PbCSP i.d. Boost: MVA.PbCSP i.d.,

No Protection

Group 2: Prime: DNA.PbCSP i.m. Boost: MVA.PbCSP i.d.

No Protection

Group 3: Prime: 10 μg Apovia i.p. Boost: 10 μg Apovia i.p.

33% protection (2 out of 6)

Group 4: Prime: 5 μg Apovia i.d. Boost: 5 μg Apovia i.d.

No Protection

Group 5: Prime: DNA.PbCSP i.m. and 10 μg Apovia i.p. Boost: 1 MVA.PbCSP admixed with 10 μg Apovia i.p.

50% protection (3 out of 6)

Group 6: Prime: FP9.PbCSP i.d. and 10 μg Apovia i.p. Boost: MVA.PbCSP i.d. and 10 μg Apovia i.p.

66% protection (4 out of 6)

Group 7: Prime: FP9.PbCSP admixed with 5 μg Apovia i.d. Boost: MVA.PbCSP admixed with 5 μg Apovia i.d.

100% protection (5 out of 5)

Group 8: Prime: PBS i.d. Boost: PBS i.d.

No Protection

Long-Term Challenge Conclusions
Immunisation with Apovia i.p. afforded 33% protection.
This was increased to 50% if DNA.PbCSP was given i.m. at the time of Apovia i.p. priming and MVA.PbCSP mixed and co-administered with Apovia i.p. at boost.
Priming with FP.PbCSP and Apovia at distinct sites(FP i.d., Apovia i.d.) and boosting with MVA.PbCSP and Apovia (FP i.d., Apovia i.d.) further increased protective efficacy to 66%.
Halving the dose of Apovia vaccine, mixing it with FP.PbCSP and MVA.PbCSP and immunizing at prime and boost induced 100% protection.

There was no strong correlation between levels of anti-(DP4NPN)2 antibodies or CD8+ Pb9 T cells, assessed individually, and protection from sporozoite challenge. However, the fully protected group, group 7 was the only group with both high level T cell responses and high level antibody titres suggesting that these may act together to produce enhanced protection.

1-57. (canceled)

58. A composition of matter comprising:
a) a viral capsid substantially incapable of replication in a patient and at least one antigen for co-administration with said capsid and against which it is desired to obtain an immune response, said composition inducing both an antibody response against the co-administered antigen and a T-cell response;
b) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid is not ALVAC;
c) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid does not comprise a polynucleotide encoding an antigen and both the T-cell and antibody responses are induced to the at least one co-administered antigen;
d) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid is not ALVAC, the viral capsid does not comprise a polynucleotide encoding an antigen, and both the T-cell and antibody responses are induced to the at least one co-administered antigen;
e) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid comprises a polynucleotide encoding a further antigen, the T-cell response being induced to the encoded antigen and the antibody response being induced to the co-administered antigen;
f) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid comprises a polynucleotide encoding a further antigen, the T-cell response being induced to the encoded antigen and the antibody response being induced to the co-administered antigen and wherein the encoded antigen and the co-administered antigen are homologous;
g) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid comprises a polynucleotide encoding a further antigen, the T-cell response being induced to the encoded antigen and the antibody response being induced to the co-administered antigen and wherein the encoded antigen and the co-administered antigen are homologous and share at least one common CD4⁺or CD8⁺ epitope; or
h) a viral capsid substantially incapable of replication in a patient and at least one antigen for co-administration with said capsid and against which it is desired to obtain an immune response, said composition inducing both an antibody response against the co-administered antigen and a T-cell response, wherein the viral capsid comprises a polynucleotide encoding a further antigen, the T-cell response being induced to the encoded antigen and the antibody response being induced to the co-administered antigen and wherein the encoded antigen and the co-administered antigen are heterologous.

59. The composition of matter according to claim 58, wherein said at least one antigen and the viral capsid are formulated separately or together and, when formulated separately, the composition being adapted to be administered as either separate formulations or as a mixture thereof.

60. The composition of matter according to claim 58, wherein the viral capsid is a viral vector.

61. The composition of matter according to claim 58, wherein the T-cell response is protective.

62. The composition of matter according to claim 58, wherein the T-cell response is induced to the co-administered antigen by the presence of the viral capsid.

63. The composition of matter according to claim 58, wherein the antibody response induced in the presence of the vector is greater than that induced by the co-administered antigen alone.

64. The composition of matter according to claim 58, wherein said capsid is: 1) a poxvirus; 2) an adenovirus; 3) MVA or NYVAC; 4) an orthopox virus; or 5) a fowlpox virus; and said co-administered antigen is derived from: 1) M. tuberculosis; 2) Plasmodium sp; 3) influenza virus; 4) HIV; 5) Hepatitis B or C virus; 6) Cytomegalovirus; 7) Human papilloma virus; 8) bacteria; 9) leishmania parasites; or 10) derived from a tumor.

65. A method for inducing an immune response against at least one antigen in an animal in need thereof, the method comprising the steps of administering a viral capsid incapable of replication in the animal and at least one antigen, wherein the at least one antigen is co-administered with the capsid.

66. The method according to claim 65, wherein the immune response comprises a combined T-cell and antibody response to the at least one co-administered antigen.

67. The method according to claim 65, wherein the immune response is a humoral response.

68. The method according to claim 65, wherein the capsid comprises a polynucleotide encoding a further antigen.

69. The method according to claim 68, wherein the encoded antigen and the co-administered antigen are homologous and the immune response comprises a combined T-cell and antibody response to the antigen.

70. The method according to claim 68, wherein the encoded further antigen and the co-administered antigen are heterologous and the immune response to the at least one co-administered antigen comprises an antibody response, the encoded antigen inducing a T-cell response thereto.

71. The method according to claim 65, wherein the viral capsid and the at least one antigen are administered substantially contemporaneously.

72. The method according to claim 65, wherein said method induces an antigen-specific T cell response to a viral capsid comprising a poxvirus-encoded antigen, the encoded antigen being heterologous to the poxvirus and comprising a source of CD4⁺ and CD8⁺ epitopes and antibodies to the co-administered antigen.

73. A method of generating an antibody response to an antigen in a vertebrate comprising co-administration, as a mixture, the antigen mixed with an orthopox virus to said vertebrate.

74. The method according to claim 73, wherein the orthopox virus is replication-impaired.

75. The method according to claim 73, wherein the orthopox virus is of the modified vaccinia virus Ankara strain or NYVAC strain or a derivative of either.

76. The method according claim 73, wherein the orthopox virus encodes the co-administered antigen or a homologous sequence.

77. The method according to claim 73, wherein the orthopox virus encodes an antigen that is heterologous to the co-administered antigen.

78. The method according to claim 73, wherein the vertebrate is a primate.

79. The method according to claim 73, wherein the orthopox virus encodes a heterologous polypeptide antigen encoding a CD4⁺ and/or CD8⁺ T cell epitope against which the vertebrate has a pre-existing specific cellular immune response that was generated by a means other than by immunization with the said recombinant orthopox virus.
